SEQUENCE LISTING (12 sequences)

SEQ ID NO: 1 - DNA, 18 bp, Artificial Sequence
  Description of Artificial Sequence Synthetic oligonucleotide
gtcttcccctccatcgtg

SEQ ID NO: 2 - DNA, 19 bp, Artificial Sequence
  Description of Artificial Sequence Synthetic oligonucleotide
catgtcgtcccagttggtg

SEQ ID NO: 3 - DNA, 20 bp, Artificial Sequence
  Description of Artificial Sequence Synthetic oligonucleotide
catcctcaccctgaagtacc

SEQ ID NO: 4 - DNA, 20 bp, Artificial Sequence
  Description of Artificial Sequence Synthetic oligonucleotide
gctgcatcttcctcttctgc

SEQ ID NO: 5 - DNA, 21 bp, Artificial Sequence
  Description of Artificial Sequence Synthetic oligonucleotide
ggggagggattctcagaaggt

SEQ ID NO: 6 - DNA, 21 bp, Artificial Sequence
  Description of Artificial Sequence Synthetic oligonucleotide
cctgcgagtttttaggaatca

SEQ ID NO: 7 - DNA, 46 bp, Artificial Sequence
  Description of Artificial Sequence Synthetic oligonucleotide
gaagattactcagagattataggatccatgggtgaagcacttgagg

SEQ ID NO: 8 - DNA, 46 bp, Artificial Sequence
  Description of Artificial Sequence Synthetic oligonucleotide
cctcaagtgcttcacccatggatcctataatctctgagtaatcttc

SEQ ID NO: 9 - DNA, 38 bp, Artificial Sequence
  Description of Artificial Sequence Synthetic oligonucleotide
tgaggttgtggaaaaagaggactatagtgaaatcatcg

SEQ ID NO: 10 - DNA, 39 bp, Artificial Sequence
  Description of Artificial Sequence Synthetic oligonucleotide
gatccgatgatttcactatagtcctctttttccacaacc

SEQ ID NO: 11 - DNA, 27 bp, Artificial Sequence
  Description of Artificial Sequence Synthetic oligonucleotide
cagttcttcctcaagtgcttcacccat

SEQ ID NO: 12 - DNA, 27 bp, Artificial Sequence
  Description of Artificial Sequence Synthetic oligonucleotide
ccctataatctctgagtaatcttcctt

(19) United States
(12) Patent Application Publication
(10) Pub. No.: US 2014/0235481 A1
(43) Pub. Date: Aug. 21, 2014

(54) CANCER BIOMARKER AND METHODS OF USE THEREOF

(71) Applicant: Rutgers, The State University of New Jersey, New Brunswick, NJ (US)

(72) Inventors: Ruth Steward, New Brunswick, NJ (US); Dale Schaar, New Brunswick, NJ (US); Svetlana Minakhina, New Brunswick, NJ (US); Dan Medina, New Brunswick, NJ (US)

(73) Assignee: Rutgers, The State University of New Jersey, New Brunswick, NJ (US)

(21) Appl. No.: 14/179,238

(22) Filed: Feb. 12, 2014

Related U.S. Application Data
(60) Provisional application No. 61/763,784, filed on Feb. 12, 2013.

(51) Int. Cl. C12Q 1/68; G01N 33/574

(52) U.S. Cl. CPC C12Q 1/6886; G01N 33/57496
USPC 506/9; 435/6.12; 435/6.11; 435/7.1

(57) ABSTRACT

Certain embodiments of the present invention provide methods for identifying subjects that have, or are at risk for developing, cancer. Certain embodiments of the present invention provide methods for determining the effectiveness of cancer treatments.

### CROSS-REFERENCE TO RELATED APPLICATION

This application claims the benefit under 35 U.S.C. §119(e) of U.S. Provisional Application No. 61/763,784, filed Feb. 12, 2013, the entire contents of which is hereby incorporated by reference.

### GOVERNMENT FUNDING

This invention was made with government support under Grant Number NIHD018055 awarded by the National Institutes of Health. The government has certain rights in the invention.

### BACKGROUND OF THE INVENTION

Cancer is a leading cause of death worldwide, accounting for 7.6 million deaths (around 13% of all deaths) in 2008 (World Health Organization, Fact Sheet No. 297, October 2011). In the United States, cancer is the second most common cause of death and accounts for nearly 1 of every 4 deaths (American Cancer Society. Cancer Facts & Figures 2012. Atlanta: American Cancer Society; 2012). The National Institutes of Health (NIH) estimates that the over-all costs of cancer in 2007 were $226.8 billion.

Hematologic cancer, which affects the blood, bone marrow and lymph nodes, includes leukemia, lymphoma and myeloma. In the United States in 2007, 119,724 people were diagnosed with a hematologic cancer, and 54,599 people died from a hematologic cancer (U.S. Cancer Statistics Working Group. United States Cancer Statistics: 1999-2007 Incidence and Mortality Web-based Report. Atlanta (Ga.): Department of Health and Human Services, Centers for Disease Control and Prevention, and National Cancer Institute; 2010). Among children and teens less than 20 years old, leukemia is the most common cancer and the leading cause of cancer death.

The presence of discrete molecular lesions has allowed the development of PCR assays that more precisely measure minimal residual disease in select hematologic malignancies. In myeloid leukemias, the Philadelphia chromosome t(9;22) in chronic myelogenous leukemia and the t(15;17) in acute promyelocytic leukemia thus far have been validated as molecular markers useful in monitoring the care of these patients. In addition, mutations in nucleophosmin 1 (NPM1), FLT3 and CCAAT/enhancer binding protein α (CEBPA α) have been shown to have clinically significant prognostic value. A potential drawback to following these mutations is they are also sometimes lost upon relapse in leukemia blasts, as an example a recent study has shown that the FLT3 mutation status changed in 22% of relapsed patients, including the loss of the FLT3 kinase domain mutation in approximately 10% of patients.

Thus, there is a clear need to identify biomarkers to identify patients with cancer and individuals at an elevated risk of developing cancer (e.g., relapsing), as well as new therapeutic targets. The ability to test the effectiveness of a potential cancer treatment is also needed.

### SUMMARY OF THE INVENTION

Accordingly, certain embodiments of the invention provide a method for identifying a subject that has cancer, or is at an elevated risk for developing cancer, comprising:

a) quantifying the level of Programmed Cell Death 2 (PDCD2) protein or RNA in a biological sample from the subject and comparing the level to a control sample; and

b) determining that the subject has cancer, or is at an elevated risk for developing cancer, based on the level of PDCD2 protein or RNA in the sample, wherein an increased level of PDCD2 protein or RNA in the sample as compared to the control indicates that the subject has cancer, or is at an elevated risk for developing cancer.

Certain embodiments of the invention provide a method for determining the effectiveness of a cancer treatment in a subject, comprising:

a) quantifying the level of Programmed Cell Death 2 (PDCD2) protein or RNA in a first biological sample from the subject before the cancer treatment;

b) quantifying the level of Programmed Cell Death 2 (PDCD2) protein or RNA in a second biological sample from the subject after the cancer treatment;

c) comparing the level in the first sample to the level in the second sample; and

d) determining that the cancer treatment is effective based on the level of PDCD2 protein or RNA in the second sample compared to the first sample, wherein a decrease in the level of PDCD2 protein or RNA in the second sample as compared to the first sample indicates that the cancer treatment is effective to reduce the cancer.

Certain embodiments of the invention provide a method for identifying a subject that has a relapse of cancer or refractory disease, or is at an elevated risk for a relapse of cancer or refractory disease, comprising:

a) quantifying the level of Programmed Cell Death 2 (PDCD2) protein or RNA in a first biological sample from the subject before a cancer treatment;

b) quantifying the level of Programmed Cell Death 2 (PDCD2) protein or RNA in a second biological sample from the subject after a cancer treatment; and

c) calculating the ratio of the level in the first sample to the level in the second sample, wherein a ratio less than about 2 indicates the subject has relapsed, or is at an elevated risk for a relapse of cancer or refractory disease.

### BRIEF DESCRIPTION OF THE FIGURES AND TABLES

FIG. 1. Functional conservation of the human and Drosophila Zfrp8/PDCD2 in development and hematopoiesis. Confocal cross-section of the lymph gland primary lobe in mid-third instar larvae (A-D). (A) Zfrp8^{null} lymph gland with scattered crystal cells throughout the gland, plasmatocytes and large lamellocytes, but no medullary zone. (B) Zfrp8 heterozygote, inner dotted line outlines medullary zone consisting of small-undifferentiated cells. (C) Expression of GFP-Zfrp8 and (D) GFP-PDCD2 in Zfrp8^{null} mutant background results in normal lymph gland organization. DNA is stained and is shown. (E) Viability of the Zfrp8^{null} mutant flies expressing GFP-Zfrp8 and GFP-PDCD2 under the control of the daughterless-GAL4 driver, compared to the viability of control mutant animals that carry a transgene or driver alone and do not express Zfrp8. *Viability is calculated as percentage of flies relative to expected.

FIG. 2. PDCD2 protein expression in human cancer cells. (A) PDCD2 protein expression in extracts from human Hodgkin's (KM-H2) and Non-Hodgkin's lymphoma (MedB-1, Karpas1106), breast (HMEC, MCF10a, and MCF7), embryonic kidney (HEK293), cervical (HeLa), leukemia (Jurkat, HL60 and ML1), fibroblast (BJ) and lung carcinoma (A549) cell lines. Controls included normal human bone marrow (BM) and peripheral blood mononuclear cells (WBC).

FIG. 3. PDCD2 protein expression in human hematopoietic progenitor cells. (A) Human CD34+ bone marrow cells were cultured in methylcellulose. Granulocyte, erythrocyte, macrophage, megakaryocyte (GEMM), granulocyte, macrophage (GM) and erythroid colonies were isolated and analyzed by Western analysis. Human bone marrow and cord blood, both unfractionated and CD34+ positive cells, were also analyzed for comparison. The human lung carcinoma cell line A549 was included as a positive control for PDCD2 expression. (B) Densitometry was utilized to normalize PDCD2 protein expression relative to the GAPDH loading control. (C) Western analysis of PDCD2 expression in normal peripheral blood (WBC 1-5) and bone marrow mononuclear cells (BM), in comparison to peripheral blood and bone marrow samples obtained from patients with acute lymphoblastic leukemia (ALL1-5) and acute myelogenous leukemia (AML1-13). The MCF7 breast carcinoma cell line (MCF7) was utilized as a positive control.

FIG. 4. PDCD2 expression in acute leukemia patients ongoing therapy. (A) PDCD2 protein expression in patient samples pre and post-treatment. Western analysis was performed on bone marrow aspirates obtained from acute leukemia and advanced myelodysplasia patients pre- (Dx) and post-treatment (Rec). Blots were probed first for PDCD2 expression, then β-actin as a loading control. (B and C) Quantitative Real Time RT-PCR was used to measure PDCD2 transcripts in blood and/or bone marrow of acute leukemia patients at diagnosis and once hematologic recovery was attained after treatment. PDCD2 transcript levels are presented relative to basal PDCD2 RNA levels present in normal unfractionated bone marrow. (B) Acute lymphoblastic leukemia (ALL) patients; (C) Acute myelogenous leukemia (AML) patients. Each RT-PCR was performed in triplicate. (Arrowheads below x axis denote relapsed/refractory patients; error bars represent the standard deviation from the mean). (D) Overall reduction in PDCD2 RNA expression in relapsed/refractory leukemia patients versus those in complete remission after induction chemotherapy (*p=0.006).

FIG. 5. PDCD2 knockdown impairs lung cancer cell proliferation. (A) Dicer substrate RNAi duplexes were transiently transfected into A549 lung carcinoma cells. PDCD2-specific Dicer substrates were directed toward PDCD2 nucleotides 768-792 (siRNA1) and nucleotides 738-762 (siRNA2) (GeneBank accession number NM_{—}002598.2). Controls included no RNA (Mock) and non-targeting RNA duplexes (Scrambled). Cell number was measured by automated trypan blue exclusion. (B) The culture viability for the indicated transfection conditions is presented as the percentage of viable cells. Each time point performed in triplicate; error bars represent the standard deviation from the mean; (*) denotes statistical significance, p<0.05. (C) Western analysis of the respective Dicer substrate RNAi duplex transfections for PDCD2 expression. Glyceraldehyde-3-phosphate dehydrogenase (GAPDH) expression was used as a loading control.

FIG. 6. Analysis of PDCD2 knockdown on cell proliferation. (A) Cells were harvested at the indicated time points from parental and PDCD2 KD cultures and cell number was measured by automated trypan blue exclusion. Each time point performed in triplicate; error bars represent the standard deviation from the mean; (*) denotes statistical significance, p<0.05. The insets show PDCD2 protein expression in the A549 and Jurkat PDCD2 KD cell lines. Cells were harvested from respective cultures that were 50-70% confluent and protein lysates prepared. Western blots were probed first for PDCD2 expression, then stripped and probed for β-actin as a loading control. (B) The first panel shows PDCD2 rescue in A549 KD cells after infection with the pMSCV-huPDCD2-shRNA^{R} retrovirus increases cell proliferation. The second panel shows partial restoration of PDCD2 protein expression by pMSCV-huPDCD2-shRNA^{R} retroviral infection by Western analysis. PDCD2 protein expression measured by densitometry and normalized to GAPDH expression. (C) DNA synthesis, as a correlate of S phase transition, was determined by EdU incorporation detected with Alexa Flour® 647 (FL-4) measured by flow cytometry. (D) PDCD2 protein expression is associated with cell proliferation. A549 cells initially seeded at 1×10⁵ per plate were cultured for the indicated time points and then cell cycle analysis was performed by propidium iodide staining followed by flow cytometry (left panel). The results presented represent the mean and standard deviations of three independent experiments. The expression of Rb, PDCD2, CDK4, CDK6, CDC2 and β-actin was analyzed in A549 at the indicated time points and increasing degrees of confluence (right panel).

FIG. 7. PDCD2 expression in lung cancer. Tissue arrays including normal lung tissue and sections from several subtypes of Lung cancer were analyzed by immunohistochemistry for PDCD2 protein expression. (C) Normal lung; (SqCC) squamous cell carcinoma; (SCC) small cell carcinoma; (LCC) large cell carcinoma; (ACC) adenocarcinoma; (AC) carcinoid; (BAC) bronchioloalveolar carcinoma. Magnification 100× and 400× as indicated.

Table 1. Prospective analysis of PDCD2 RNA expression: Patient Characteristics.

Table 2. Evaluation of PDCD2 subcellular localization between normal controls and lung cancer samples.

### DETAILED DESCRIPTION

Certain embodiments of the present invention provide methods for identifying a subject that has cancer, or is at an elevated risk for developing cancer, comprising determining the level of expression of Programmed Cell Death 2 (PDCD2) in a biological sample from the subject, wherein an increased level of expression, as compared to a control, indicates that the subject has cancer, or is at an elevated risk for developing cancer.

Certain embodiments of the present invention provide a method for identifying a subject that has cancer, or is at an elevated risk for developing cancer, comprising:

a) quantifying the level of Programmed Cell Death 2 (PDCD2) protein or RNA in a biological sample from the subject and comparing the levels to a control sample; and

b) determining that the subject has cancer, or is at an elevated risk for developing cancer, based on the level of PDCD2 protein or RNA in the sample, wherein an increased level of PDCD2 protein or RNA in the sample as compared to the control indicates that the subject has cancer, or is at an elevated risk for developing cancer.

A method for identifying a subject that has cancer, or is at an elevated risk for developing cancer, comprising:

a) obtaining a biological sample from the subject;

b) quantifying the level of Programmed Cell Death 2 (PDCD2) protein or RNA in the biological sample and comparing the level to a control sample; and

c) determining that the subject has cancer, or is at an elevated risk for developing cancer, based on the level of PDCD2 protein or RNA in the sample, wherein an increased level of PDCD2 protein or RNA in the sample as compared to the control indicates that the subject has cancer, or is at an elevated risk for developing cancer.

Certain embodiments of the invention provide determining the effectiveness of a cancer treatment in a subject, comprising:

a) quantifying the level of Programmed Cell Death 2 (PDCD2) protein or RNA in a first biological sample from the subject before the cancer treatment;

b) quantifying the level of Programmed Cell Death 2 (PDCD2) protein or RNA in a second biological sample from the subject after the cancer treatment;

c) comparing the level in the first sample to the level in the second sample; and

d) determining that the cancer treatment is effective based on the level of PDCD2 protein or RNA in the second sample compared to the first sample, wherein a decrease in the level of PDCD2 protein or RNA in the second sample as compared to the first sample indicates that the cancer treatment is effective to reduce the cancer.

Certain embodiments of the invention provide, a method for determining the effectiveness of a cancer treatment in a subject, comprising:

a) obtaining a first biological sample from the subject before the cancer treatment;

b) quantifying the level of Programmed Cell Death 2 (PDCD2) protein or RNA in the first biological sample;

c) obtaining a second biological sample from the subject after the cancer treatment;

d) quantifying the level of Programmed Cell Death 2 (PDCD2) protein or RNA in the second biological sample;

e) comparing the level in the first sample to the level in the second sample; and

f) determining that the cancer treatment is effective based on the level of PDCD2 protein or RNA in the second sample compared to the first sample, wherein a decrease in the level of PDCD2 protein or RNA in the second sample as compared to the first sample indicates that the cancer treatment is effective to reduce the cancer.

Certain embodiments of the invention provide a method for identifying a subject that has a relapse of cancer or refractory disease, or is at an elevated risk for a relapse of cancer or refractory disease, comprising:

a) quantifying the level of Programmed Cell Death 2 (PDCD2) protein or RNA in a first biological sample from the subject before a cancer treatment;

b) quantifying the level of Programmed Cell Death 2 (PDCD2) protein or RNA in a second biological sample from the subject after a cancer treatment; and

c) calculating the ratio of the level in the first sample to the level in the second sample, wherein a ratio less than about 3.5 indicates the subject has relapsed or has refractory disease, or is at an elevated risk for a relapse of cancer or refractory disease.

Certain embodiments of the invention provide, a method for identifying a subject that has a relapse of cancer or has refractory disease, or is at an elevated risk for a relapse of cancer or refractory disease, comprising

a) obtaining a first biological sample from the subject before a cancer treatment;

b) quantifying the level of Programmed Cell Death 2 (PDCD2) protein or RNA in the first biological sample from the subject;

c) obtaining a second biological sample from the subject after a cancer treatment;

d) quantifying the level of Programmed Cell Death 2 (PDCD2) protein or RNA in the second biological sample from the subject; and

e) calculating the ratio of the level in the first sample to the level in the second sample, wherein a ratio less than about 3.5 indicates the subject has relapsed or has refractory disease, or is at an elevated risk for a relapse of cancer or refractory disease.

In certain embodiments, a ratio less than about 3 indicates the subject has relapsed or has refractory disease, or is at an elevated risk for a relapse of cancer or refractory disease. In certain embodiments, a ratio less than about 2 indicates the subject has relapsed or has refractory disease, or is at an elevated risk for a relapse of cancer or refractory disease. In certain embodiments, a ratio less than about 1.7 indicates the subject has relapsed or has refractory disease, or is at an elevated risk for a relapse of cancer or refractory disease. In certain embodiments, a ratio of about 1 indicates the subject has relapsed or has refractory disease, or is at an elevated risk for a relapse of cancer or refractory disease.

Certain embodiments of the invention provide, a method to determine whether to administer a cancer treatment to a subject, comprising:

a) quantifying the level of Programmed Cell Death 2 (PDCD2) protein or RNA in a biological sample from the subject and comparing the level to a control sample; and

c) determining that a cancer treatment should be administered to the subject based on the level of PDCD2 protein or RNA in the sample, wherein an increased level of PDCD2 protein or RNA in the sample as compared to the control indicates that the cancer treatment should be administered.

Certain embodiments of the invention provide a method to determine whether to administer a cancer treatment to a subject, comprising:

a) obtaining a biological sample from the subject;

b) quantifying the level of Programmed Cell Death 2 (PDCD2) protein or RNA in the biological sample and comparing the level to a control sample; and

c) determining that a cancer treatment should be administered to the subject based on the level of PDCD2 protein or RNA in the sample, wherein an increased level of PDCD2 protein or RNA in the sample as compared to the control indicates that the cancer treatment should be administered.

Certain embodiments of the invention provide, a method to determine whether to administer additional cancer treatment to a subject, comprising:

a) quantifying the level of Programmed Cell Death 2 (PDCD2) protein or RNA in a first biological sample from the subject before an initial cancer treatment;

b) quantifying the level of Programmed Cell Death 2 (PDCD2) protein or RNA in a second biological sample from the subject after the initial cancer treatment;

c) determining the ratio of the level in the first sample to the level in the second sample, wherein a ratio less than about 3 indicates that additional and/or a change in cancer treatment should be administered.

Certain embodiments of the invention provide a method to determine whether to administer additional cancer treatment to a subject, comprising:

a) obtaining a first biological sample from the subject before an initial cancer treatment;

b) quantifying the level of Programmed Cell Death 2 (PDCD2) protein or RNA in the first biological sample from the subject;

c) obtaining a second biological sample from the subject after the initial cancer treatment;

d) quantifying the level of Programmed Cell Death 2 (PDCD2) protein or RNA in a second biological sample from the subject;

e) determining the ratio of the level in the first sample to the level in the second sample, wherein a ratio less than about 3 indicates that additional and/or a change in cancer treatment should be administered.

In certain embodiments, a ratio less than about 2 indicates that additional cancer treatment should be administered.

In certain embodiments, a ratio of about 1 indicates that additional cancer treatment should be administered.

Certain embodiments of the invention provide a method for identifying a subject that has cancer, or is at an elevated risk for developing cancer, comprising

(a) transporting a biological sample from the subject suspected of having or being at an elevated risk for developing cancer to a diagnostic laboratory;

(b) detecting and quantifying the level of Programmed Cell Death 2 (PDCD2) protein or RNA in the biological sample, and comparing the level to a control sample;

(c) identifying that the subject has cancer, or is at an elevated risk for developing cancer, when the sample has an increased level of PDCD2 protein or RNA as compared to the control; and

(d) providing results regarding whether the subject has cancer, or is at an elevated risk for developing cancer.

Certain embodiments of the present invention provide a method for identifying a subject that has cancer, or is at an elevated risk for developing cancer, comprising:

a) obtaining a biological sample from the subject;

b) examining Programmed Cell Death 2 (PDCD2) cellular localization in the biological sample and comparing the localization to a control sample; and

c) determining that the subject has cancer, or is at an elevated risk for developing cancer, based on the cellular localization of PDCD2, wherein a loss of nuclear localization of PDCD2 as compared to the control indicates that the subject has cancer, or is at an elevated risk for developing cancer.

Techniques and assays for examining the cellular localization of a protein in a biological sample (e.g., a tissue sample or a sample comprising cells) is known in the art, and may include, for example, examining the sample by immunohistochemistry or fluorescence. In certain embodiments, the cellular localization of PDCD2 may be examined by contacting the sample with an antibody. In certain embodiments, the antibody is a PDCD2 antibody (e.g., a monoclonal or polyclonal PDCD2 antibody). In certain embodiments, the antibody binds to PDCD2. In certain embodiments, the methods further comprise contacting the sample with a secondary antibody. In certain embodiments, the antibody or secondary antibody is labeled (e.g., with a fluorophore).

In certain embodiments of the invention, the subject has, or is at an elevated risk for developing cancer. In certain embodiments, the subject has a relapse of cancer or has refractory disease, or is at an elevated risk for a relapse of cancer or refractory disease.

In certain embodiments, the subject is at an elevated risk for developing cancer.

In certain embodiments, the subject has cancer.

In certain embodiments, the subject has a relapse of cancer.

In certain embodiments, the subject has refractory disease.

In certain embodiments of the invention, the subject is an elevated risk of suffering a relapse of cancer or refractory disease.

In certain embodiments, the subject has normal cytogenetics.

In certain embodiments, the cancer is acute leukemia.

In certain embodiments, the cancer is a hematologic malignancy. In certain embodiments, the hematologic malignancy is selected from acute lymphoblastic leukemia, acute myelogenous leukemia, chronic lymphocytic leukemia, chronic myelogenous leukemia, advanced myelodysplasia, multiple myeloma and lymphoma.

In certain embodiments, the cancer is a carcinoma. In certain embodiments, the carcinoma is breast, ovarian, lung, gastrointestinal (e.g., colon), or genitourinary (e.g., renal, prostate or testicular) carcinoma. In certain embodiments, the cancer is lung cancer.

Biological samples may be obtained through techniques well known in the art. For example, in certain embodiments, the sample is peripheral blood, which may be obtained by, e.g., drawing blood out of the subject's vein with a needle. In certain embodiments, the blood sample comprises detectable leukemic blasts by standard laboratory measurements. In certain embodiments, the blood sample comprises at least about 5, 10, 20, 30, 40, 50, 60, 70, 80, 90 or 95 percent leukemic blasts. In certain embodiments, the blood sample comprises at least about 70 percent leukemic blasts. In certain embodiments, the blood sample comprises no detectable leukemic blasts by standard laboratory measurements.

In certain embodiments, the sample is bone marrow, which may be obtained by, e.g., drawing bone marrow out of the subject's bone with a needle. In certain embodiments, the sample is bone marrow mononuclear cells. In certain embodiments, the bone marrow is unfractionated.

In certain embodiments, the sample is a tissue sample (e.g., lung tissue), which may be obtained, e.g., from a biopsy.

In certain embodiments, the sample is taken after administration of a cancer treatment.

In certain embodiments of the invention, the methods comprise quantifying the level of Programmed Cell Death 2 (PDCD2) protein or RNA in a biological sample(s).

In certain embodiments, the method comprises quantifying the levels of PDCD2 RNA. In certain embodiments, the method comprises quantifying the levels of PDCD2 mRNA. Methods and techniques for quantifying the level of an RNA in a biological sample(s) are known in the art, for example, in certain embodiments, the level of PDCD2 RNA is quantified by reverse transcription polymerase chain reaction (RT-PCR), quantitative Real Time RT-PCR, Northern blot analysis, expression microarray analysis or next generation sequencing (NGS). In certain embodiments, the level of PDCD2 RNA is quantified by quantitative Real Time RT-PCR.

For example, in certain embodiments of the invention, quantifying the levels of PDCD2 RNA in the biological sample may comprise first isolating RNA from the biological sample(s) (e.g., the biological sample or the first and/or second biological sample). In certain embodiments, the RNA is mRNA. In certain embodiments, the RNA is PDCD2 mRNA. Methods and techniques for isolating RNA from a biological sample(s) are known in the art, for example, through the use of Trizol as described in the Examples.

In certain embodiments of the invention, the methods further comprise reverse transcribing mRNA isolated from the biological sample to generate cDNA. In certain embodiments, the methods further comprise reverse transcribing PDCD2 mRNA isolated from the biological sample to generate PDCD2 cDNA.

In certain embodiments of the invention, quantifying the levels of PDCD2 RNA in the biological sample may comprise contacting the sample with at least one oligonucleotide probe to form a hybridized nucleic acid. In certain embodiments, the at least one oligonucleotide probe is immobilized on a solid surface. In certain embodiments of the invention, the methods further comprise contacting the sample with a first oligonucleotide probe to form a first hybridized nucleic acid and contacting the sample with a second oligonucleotide probe to form a second hybridized nucleic acid.

In certain embodiments of the invention, quantifying the levels of PDCD2 RNA in the biological sample may comprise contacting PDCD2 cDNA, e.g., generated from mRNA isolated from the biological sample, with at least one oligonucleotide probe to form a hybridized nucleic acid. In certain embodiments, the at least one oligonucleotide probe is immobilized on a solid surface. In certain embodiments of the invention, the methods further comprise contacting the sample with a first oligonucleotide probe to form a first hybridized nucleic acid and contacting the sample with a second oligonucleotide probe to form a second hybridized nucleic acid.

In certain embodiments, the methods further comprise amplifying the hybridized nucleic acids. In certain embodiments, amplification of the hybridized nucleic acid is carried out by, e.g., polymerase chain reaction. In certain embodiments, the hybridized nucleic acid(s) comprises a sequence which corresponds to a PDCD2 sequence (see, e.g., GenBank Accession No. NM_{—}002598.3 or GenBank Accession No. NM_{—}002598.2). In certain embodiments, the oligonucleotide probe(s) is complementary to a PDCD2 sequence. In certain embodiments, the oligonucleotide probe(s) comprises a sequence selected from (5′ to 3′) GCTGCATCTTCCTCTTCTGC (SEQ ID NO:4) and GGGGAGGGATTCTCAGAAGGT (SEQ ID NO:5). In certain embodiments, the oligonucleotide probe(s) shares 99, 95, 90, 85, 80, 75, 70, 65, 60, 55, 50% sequence identity with (5′ to 3′) GCTGCATCTTCCTCTTCTGC (SEQ ID NO:4) or GGGGAGGGATTCTCAGAAGGT (SEQ ID NO:5). In certain embodiments, the oligonucleotide probe(s) is selected from (5′ to 3′) GCTGCATCTTCCTCTTCTGC (SEQ ID NO:4) and GGGGAGGGATTCTCAGAAGGT (SEQ ID NO:5).

In certain embodiments, the methods further comprise contacting the amplified nucleic acid(s) with a detection oligonucleotide probe, wherein the detection oligonucleotide probe hybridizes to the amplified nucleic acid(s). In certain embodiments, the detection oligonucleotide probe is complementary to a PDCD2 sequence. In certain embodiments, the detection oligonucleotide probe comprises CCTGCGAGTTTTTAGGAATCA (SEQ ID NO:6). In certain embodiments, the detection oligonucleotide probe shares a 99, 95, 90, 85, 80, 75, 70, 65, 60, 55, 50% sequence identity with CCTGCGAGTTTTTAGGAATCA (SEQ ID NO:6). In certain embodiments, the detection oligonucleotide probe is CCTGCGAGTTTTTAGGAATCA (SEQ ID NO:6). In certain embodiments, the detection oligonucleotide probe is labeled (e.g., with a fluorophore or a radio-isotope). In certain embodiments, the detection probe is labeled with a reporter fluorophore. In certain embodiments, the detection probe is labeled with a reporter fluorophore and a quencher fluorophore, wherein the reporter fluorophore emits at a wavelength absorbed by the quencher fluorophore. In certain embodiments, the detection oligonucleotide probe is (5′-6-FAM) CCTGCGAGTTTTTAGGAATCA (3′Iowa Black® FQ) (SEQ ID NO:6).

In certain other embodiments, the methods comprise quantifying the level of PDCD2 protein. Methods and techniques for quantifying the level of a protein in a biological sample(s) are known in the art, for example, in certain embodiments, the level of PDCD2 protein is quantified by Western analysis or immunohistochemistry of tissue arrays.

For example, in certain embodiments of the invention, quantifying the level of PDCD2 protein in the biological sample may further comprise isolating protein from the biological sample(s) (e.g., the biological sample or the first and/or second biological sample). Methods and techniques for isolating protein from a biological sample(s) are known in the art, for example, as described in the Examples, cell lysates derived from the sample are generated, denatured and separated on a polyacrylamide gel. In certain embodiments, the proteins are transferred to a membrane.

In certain embodiments of the invention, quantifying the level of PDCD2 protein in the biological sample comprises contacting the sample with an antibody. In certain embodiments of the invention, quantifying the level of PDCD2 protein in the biological sample comprises contacting a cell from the sample with an antibody. In certain embodiments of the invention, quantifying the level of PDCD2 protein in the biological sample comprises contacting proteins isolated from the sample with an antibody. In certain embodiments, the antibody is a PDCD2 antibody. In certain embodiments, the PDCD2 antibody is a monoclonal antibody. In certain embodiments, the PDCD2 antibody is a polyclonal antibody. In certain embodiments, the methods further comprise contacting the sample with a secondary antibody. In certain embodiments, the antibody or secondary antibody is labeled (e.g., with a fluorophore).

In certain embodiments, level of PDCD2 protein or RNA in the biological sample is normalized to the level of a control protein or RNA in the biological sample. In certain embodiments, densitometry is used to normalize the level of PDCD2 protein or RNA in the biological sample to that of a control protein or RNA.

In certain embodiments, the control protein is beta-actin, Glyceraldehyde-3-phosphate dehydrogenase (GAPDH) or ubiquitin. In certain embodiments, the control RNA encodes beta-actin, GAPDH or ubiquitin.

In certain embodiments of the invention, the methods further comprise administering a cancer treatment to the subject. In certain embodiments, the methods further comprise administering an initial cancer treatment to the subject. Cancer treatments are well known in the art and may include, e.g., chemotherapy and/or stem cell transplantation and/or radiation. In certain embodiments, the cancer treatment inhibits the expression of Programmed Cell Death 2 (PDCD2) in the subject.

Certain embodiments of the invention also provide a method to treat cancer comprising inhibiting the expression of Programmed Cell Death 2 (PDCD2) in a subject. In certain embodiments, inhibiting the expression of PDCD2 in a subject impairs cancer cell proliferation.

In certain embodiments, the expression of PDCD2 is inhibited using RNA interference. RNA interference methods are known to the skilled artisan, for example, in certain embodiments, the RNA interference is siRNA or shRNA (e.g., as described in the Examples). In certain embodiments, the RNA interference is siRNA. In certain embodiments, the RNA interference is shRNA.

Certain embodiments of the present invention provide kits for identifying a subject that has cancer, or is at an elevated risk for developing cancer. These kits contain packaging material, at least one reagent for quantifying the level of Programmed Cell Death 2 (PDCD2) protein or RNA in a biological sample from the subject, and instructions for its intended use.

Certain embodiments of the invention provide a kit for identifying a subject that has cancer, or is at an elevated risk for developing cancer, comprising at least one reagent for quantifying the level of Programmed Cell Death 2 (PDCD2) protein or RNA in a biological sample from a subject; and instructions for quantifying the level of Programmed Cell Death 2 (PDCD2) protein or RNA in the biological sample and to compare the level to a control sample, wherein an increased level of PDCD2 protein or RNA in the sample as compared to the control indicates that the subject has cancer, or is at an elevated risk for developing cancer.

Certain embodiments of the invention provide a kit for identifying a subject that has cancer, or is at an elevated risk for developing cancer, comprising at least one reagent as described herein for quantifying the level of Programmed Cell Death 2 (PDCD2) RNA in a biological sample from a subject; and instructions for quantifying the level of Programmed Cell Death 2 (PDCD2) RNA in the biological sample and to compare the level to a control sample, wherein an increased level of PDCD2 RNA in the sample as compared to the control indicates that the subject has cancer, or is at an elevated risk for developing cancer. In certain embodiments, the reagent is an oligonucleotide probe complementary to a PDCD2 sequence (see, e.g., GenBank Accession No. NM_{—}002598.3 or GenBank Accession No. NM_{—}002598.2). In certain embodiments, the oligonucleotide probe(s) comprises a sequence selected from (5′ to 3′) GCTGCATCTTCCTCTTCTGC (SEQ ID NO:4) and GGGGAGGGATTCTCAGAAGGT (SEQ ID NO:5). In certain embodiments, the oligonucleotide probe(s) shares 99, 95, 90, 85, 80, 75, 70, 65, 60, 55, 50% sequence identity with (5′ to 3′) GCTGCATCTTCCTCTTCTGC (SEQ ID NO:4) or GGGGAGGGATTCTCAGAAGGT (SEQ ID NO:5). In certain embodiments, the oligonucleotide probe(s) is selected from (5′ to 3′) GCTGCATCTTCCTCTTCTGC (SEQ ID NO:4) and GGGGAGGGATTCTCAGAAGGT (SEQ ID NO:5). In certain embodiments, the reagent is a detection oligonucleotide probe. In certain embodiments, the detection oligonucleotide probe is complementary to a PDCD2 sequence. In certain embodiments, the detection oligonucleotide probe comprises CCTGCGAGTTTTTAGGAATCA (SEQ ID NO:6). In certain embodiments, the detection oligonucleotide probe shares 99, 95, 90, 85, 80, 75, 70, 65, 60, 55, 50% sequence identity with CCTGCGAGTTTTTAGGAATCA (SEQ ID NO:6). In certain embodiments, the detection oligonucleotide probe is CCTGCGAGTTTTTAGGAATCA (SEQ ID NO:6). In certain embodiments, the detection oligonucleotide probe is labeled (e.g., with a fluorophore or a radio-isotope). In certain embodiments, the detection probe is labeled with a reporter fluorophore. In certain embodiments, the detection probe is labeled with a reporter fluorophore and a quencher fluorophore, wherein the reporter fluorophore emits at a wavelength absorbed by the quencher fluorophore. In certain embodiments, the detection oligonucleotide probe is (5′-6-FAM) CCTGCGAGTTTTTAGGAATCA (3′Iowa Black® FQ) (SEQ ID NO:6).

Certain embodiments of the invention provide a kit for identifying a subject that has cancer, or is at an elevated risk for developing cancer, comprising at least one reagent for quantifying the level of Programmed Cell Death 2 (PDCD2) protein in a biological sample from a subject; and instructions for quantifying the level of Programmed Cell Death 2 (PDCD2) protein in the biological sample and to compare the level to a control sample, wherein an increased level of PDCD2 protein in the sample as compared to the control indicates that the subject has cancer, or is at an elevated risk for developing cancer. In certain embodiments, the reagent is an antibody (e.g., a PDCD2 antibody).

As used herein, the terms “treat” and “treatment” can refer to therapeutic treatment and prophylactic or preventative treatment. In some embodiments of the invention, the object is to prevent or decrease the development of cancer. Those subjects in need of treatment include those having cancer and those having a predisposition to developing cancer. Accordingly, certain embodiments of the invention relate to determining the effectiveness of a cancer treatment.

The terms “cancer” and “cancerous” refer to the physiological condition in mammals that is typically characterized by unregulated cell growth. Examples of cancers include, but are not limited to, carcinomas (e.g., breast, ovarian, lung, gastrointestinal (e.g., colon), or genitourinary (e.g., renal, prostate or testicular)) and hematologic malignancies, such as leukemia, lymphoma, and myeloma.

As used herein, the term “control” refers to a biological sample from a subject that does not have cancer and is not at an elevated risk for developing cancer.

As used herein, the phrase “control protein or RNA” can refer to a protein or RNA whose expression remains constant and is not affected by cancer. In certain embodiments, the control protein or RNA is encoded by a housekeeping gene, for example, a gene that produces proteins such as actin, GAPDH, or ubiquitin. For example, in certain embodiments, the control protein is beta-actin or the control RNA encodes beta-actin.

As used herein, the phrase “is at an elevated risk for developing cancer” can refer to a subject at an elevated risk for initially developing cancer or at an elevated risk for relapse.

The term “relapse” as used herein describes the return of the signs and symptoms of disease following a period of remission or other improvements. In certain embodiments, relapse occurs at least within about 5 years from completion of initial treatment. Remission can describe disease that was once present but is no longer detected and an absence of signs and symptoms. In certain embodiments, for example, a subject may be at an elevated risk for refractory disease. Refractory disease refers to disease that does not respond to therapy, for example, relapsing within about 6 months (e.g., about 5, 4, 3, 2, or 1) of initial treatment (e.g., induction chemotherapy).

As used herein, the phrase “has cancer” can refer to a subject that has detectable cancer and signs and symptoms of cancer or to a subject wherein signs and symptoms of cancer have disappeared but cancerous cells are still present in the subject. In certain embodiments, minimal residual disease in a subject may be monitored using the methods described herein.

As used herein, the term “subject” refers to a mammal, e.g., a human. In certain embodiments, the subject is female and in certain embodiments the subject is male.

The term “biomarker” is generally defined herein as a biological indicator, such as a particular molecular feature, that may affect or be related to diagnosing or predicting an individual's health.

“Oligonucleotide probe” can refer to a nucleic acid segment, such as a primer, that may be useful to amplify a sequence in the nucleic acid of interest (e.g., PDCD2 RNA, mRNA or cDNA) and that is complementary to, and hybridizes specifically to, a particular sequence in the nucleic acid of interest.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

In one embodiment of the present invention, the method also involves contacting the sample with at least one oligonucleotide probe to form a hybridized nucleic acid and amplifying the hybridized nucleic acid. “Amplifying” utilizes methods such as the polymerase chain reaction (PCR), ligation amplification (or ligase chain reaction, LCR), strand displacement amplification, nucleic acid sequence-based amplification, and amplification methods based on the use of Q-beta replicase. These methods are well known and widely practiced in the art. Reagents and hardware for conducting PCR are commercially available. In another embodiment of the present invention, at least one oligonucleotide probe is immobilized on a solid surface.

As used herein, the term “specifically hybridizes” or “specifically detects” refers to the ability of a nucleic acid molecule to hybridize to at least approximately six consecutive nucleotides of a sample nucleic acid.

The terms “protein,” “peptide” and “polypeptide” are used interchangeably herein.

In the context of the present invention, an “isolated” or “purified” nucleic acid molecule is a molecule that, by human intervention, exists apart from its native environment. An isolated nucleic acid molecule may exist in a purified form or may exist in a non-native environment. For example, an “isolated” or “purified” nucleic acid molecule, or portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences and proteins or partial-length proteins encoded thereby are also encompassed by the present invention.

By “fragment” or “portion” of a sequence is meant a full length or less than full length of the nucleotide sequence encoding, or the amino acid sequence of a polypeptide or protein. As it relates to a nucleic acid molecule, sequence or segment of the invention when linked to other sequences for expression, “portion” or “fragment” means a sequence having, for example, at least 80 nucleotides, at least 150 nucleotides, or at least 400 nucleotides. If not employed for expressing, a “portion” or “fragment” means, for example, at least 9, 12, 15, or at least 20, consecutive nucleotides, e.g., probes and primers (oligonucleotides), corresponding to the nucleotide sequence of the nucleic acid molecules of the invention. Alternatively, fragments or portions of a nucleotide sequence that are useful as hybridization probes generally do not encode fragment proteins retaining biological activity. Thus, fragments or portions of a nucleotide sequence may range from at least about 6 nucleotides, about 9, about 12 nucleotides, about 20 nucleotides, about 50 nucleotides, about 100 nucleotides or more.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98%, to 99% sequence identity to the native (endogenous) nucleotide sequence.

“Synthetic” polynucleotides are those prepared by chemical synthesis.

“Recombinant nucleic acid molecule” is a combination of nucleic acid sequences that are joined together using recombinant DNA technology and procedures used to join together DNA sequences as described, for example, in Sambrook and Russell (2001).

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, such as PDCD2, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation.

“Somatic mutations” are those that occur only in certain tissues, e.g., in liver tissue, and are not inherited in the germline. “Germline” mutations can be found in any of a body's tissues and are inherited.

“Homology” refers to the percent identity between two polynucleotides or two polypeptide sequences. Two DNA or polypeptide sequences are “homologous” to each other when the sequences exhibit at least about 75% to 99% (including 75%, 76%, 77%, 78%, 79%, 80%, 81%, 82%, 83%, 84%, 85%, 86%. 87%, 88%, 89%, 90%, 91%, 92%, 93%, 94%, 95%, 96%, 97%, 98%, 99%) contiguous sequence identity over a defined length of the sequences.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information (see the World Wide Web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When using BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the World Wide Web at ncbi.nlm.nih.gov. Alignment may also be performed manually by visual inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by a BLAST program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, or at least 80%, 90%, or even at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%; at least 80%, 81%, 82%, 83%, 84%, 85%, 86%, 87%, 88%, or 89%; or at least 90%, 91%, 92%, 93%, or 94%; or even at least 95%, 96%, 97%, 98% or 99% sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl:

Tₘ81.5° C.+16.6(log M)+0.41(% GC)−0.61(% form)−500/L

where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. Guidance as to appropriate amino acid substitutions that do not affect biological activity of the protein of interest are well known in the art. Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally-occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations.”

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Coding sequence” refers to a DNA or RNA sequence that codes for a specific amino acid sequence and excludes the non-coding sequences. For example, a DNA “coding sequence” or a “sequence encoding” a particular polypeptide, is a DNA sequence which is transcribed and translated into a polypeptide in vitro or in vivo when placed under the control of appropriate regulatory elements. The boundaries of the coding sequence are determined by a start codon at the 5′-terminus and a translation stop codon at the 3′-terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence. It may constitute an “uninterrupted coding sequence,” i.e., lacking an intron, such as in a cDNA or it may include one or more introns bounded by appropriate splice junctions. An “intron” is a sequence of RNA that is contained in the primary transcript but that is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

The term “regulatory sequence” is art-recognized and intended to include promoters, enhancers and other expression control elements (e.g., polyadenylation signals). Such regulatory sequences are known to those skilled in the art. It should be understood that the design of the expression vector may depend on such factors as the choice of the host cell to be transfected and/or the amount of fusion protein to be expressed.

The term DNA “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

A control element, such as a promoter, “directs the transcription” of a coding sequence in a cell when RNA polymerase binds the promoter and transcribes the coding sequence into mRNA, which is then translated into the polypeptide encoded by the coding sequence.

“Transcription stop fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

“Translation stop fragment” or “translation stop codon” or “stop codon” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. The change of at least one nucleotide in a nucleic acid sequence can result in an interruption of the coding sequence of the gene, e.g., a premature stop codon.

According to the methods of the present invention, the amplification of nucleic acids present in a physiological sample may be carried out by any means known to the art. Examples of suitable amplification techniques include, but are not limited to, polymerase chain reaction (including, for RNA amplification, reverse-transcriptase polymerase chain reaction), ligase chain reaction, strand displacement amplification, transcription-based amplification, self-sustained sequence replication (or “3SR”), the Qβ replicase system, nucleic acid sequence-based amplification (or “NASBA”), the repair chain reaction (or “RCR”), and boomerang DNA amplification (or “BDA”).

The bases incorporated into the amplification product may be natural or modified bases (modified before or after amplification), and the bases may be selected to optimize subsequent electrochemical detection steps.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable nucleic acid polymerase) with one oligonucleotide primer for each strand of the specific sequence to be detected under hybridizing conditions so that an extension product of each primer is synthesized that is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe described herein), the probe carrying a detectable label, and then detecting the label in accordance with known techniques. Where the nucleic acid to be amplified is RNA, amplification may be carried out by initial conversion to DNA by reverse transcriptase in accordance with known techniques.

Strand displacement amplification (SDA) may be carried out in accordance with known techniques. For example, SDA may be carried out with a single amplification primer or a pair of amplification primers, with exponential amplification being achieved with the latter. In general, SDA amplification primers comprise, in the 5′ to 3′ direction, a flanking sequence (the DNA sequence of which is noncritical), a restriction site for the restriction enzyme employed in the reaction, and an oligonucleotide sequence (e.g., an oligonucleotide probe of the present invention) that hybridizes to the target sequence to be amplified and/or detected. The flanking sequence, which serves to facilitate binding of the restriction enzyme to the recognition site and provides a DNA polymerase priming site after the restriction site has been nicked, is about 15 to 20 nucleotides in length in one embodiment. The restriction site is functional in the SDA reaction. The oligonucleotide probe portion is about 13 to 15 nucleotides in length in one embodiment of the invention.

Ligase chain reaction (LCR) is also carried out in accordance with known techniques. In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

Diagnostic techniques that are useful in the methods of the invention include, but are not limited to direct DNA sequencing, PFGE analysis, allele-specific oligonucleotide (ASO), dot blot analysis and denaturing gradient gel electrophoresis, and are well known to the artisan.

Nucleic acid analysis via microchip and microarray technology is also applicable to the present invention.

As noted above, the methods of the present invention is useful for detecting and quantifying the level of a protein or mRNA in a sample.

Oligonucleotide probes may be prepared having any of a wide variety of base sequences according to techniques that are well known in the art. Suitable bases for preparing the oligonucleotide probe may be selected from naturally occurring nucleotide bases such as adenine, cytosine, guanine, uracil, and thymine; and non-naturally occurring or “synthetic” nucleotide bases such as 7-deaza-guanine 8-oxo-guanine, 6-mercaptoguanine, 4-acetylcytidine, 5-(carboxyhydroxyethyl)uridine, 2′-O-methylcytidine, 5-carboxymethylamino-methyl-2-thioridine, 5-carboxymethylaminomethyluridine, dihydrouridine, 2′-O-methylpseudouridine, β,D-galactosylqueosine, 2′-O-methylguanosine, inosine, N6-isopentenyladenosine, 1-methyladenosine, 1-methylpseeudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylamninomethyluridine, 5-methoxyaminomethyl-2-thiouridine, β,D-mannosylqueosine, 5-methloxycarbonylmethyluridine, 5-methoxyuridine, 2-methyltio-N6-isopentenyladenosine, N-((9-β-D-ribofuranosyl-2-methylthiopurine-6-yl)carbamoyl)threonine, N-((9-β-D-ribofuranosylpurine-6-yl)N-methyl-carbamoyl)threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-Methylurdine, N-((9-beta-D-ribofuranosylpurine-6-yl)carbamoyl)threonine, 2′-O-methyl-5-methyluridine, 2′-O-methylurdine, wybutosine, and 3-(3-amino-3-carboxypropyl)uridine. Any oligonucleotide backbone may be employed, including DNA, RNA (although RNA is less preferred than DNA), modified sugars such as carbocycles, and sugars containing 2′ substitutions such as fluoro and methoxy. The oligonucleotides may be oligonucleotides wherein at least one, or all, of the internucleotide bridging phosphate residues are modified phosphates, such as methyl phosphonates, methyl phosphonotlioates, phosphoroinorpholidates, phosphoropiperazidates and phosplioramidates (for example, every other one of the internucleotide bridging phosphate residues may be modified as described). The oligonucleotide may be a “peptide nucleic acid” such as described in Nielsen et al., Science, 254, 1497-1500 (1991).

The only requirement is that the oligonucleotide probe should possess a sequence at least a portion of which is capable of binding to a known portion of the nucleic acid sequence of interest (e.g., PDCD2).

It may be desirable in some applications to contact the nucleic acid sample with a number of oligonucleotide probes having different base sequences (e.g., where there are two or more target nucleic acids in the sample, or where a single target nucleic acid is hybridized to two or more probes in a “sandwich” assay).

The nucleic acid probes provided by the present invention are useful for a number of purposes. The probes can be used to detect PCR amplification products.

The nucleic acid sample may be contacted with the oligonucleotide probe in any suitable manner known to those skilled in the art. For example, the nucleic acid sample may be solubilized in solution, and contacted with the oligonucleotide probe by solubilizing the oligonucleotide probe in solution with the nucleic acid sample under conditions that permit hybridization. Suitable conditions are well known to those skilled in the art. Alternatively, the nucleic acid sample may be solubilized in solution with the oligonucleotide probe immobilized on a solid support, whereby the nucleic acid sample may be contacted with the oligonucleotide probe by immersing the solid support having the oligonucleotide probe immobilized thereon in the solution containing the nucleic acid sample.

The invention will now be illustrated by the following non-limiting Examples.

### Example 1

Programmed Cell Death 2 (PDCD2) facilitates cancer cell growth and is highly expressed in acute leukemia. PDCD2 is an evolutionarily conserved eukaryotic protein with unknown function. The Drosophlia PDCD2 ortholog Zinc finger protein RP-8 (Zfrp8) has an essential function in fly hematopoiesis. Zfrp8 mutants exhibit a developmental delay, lethality during larval and pupal stages and marked lymph gland hyperplasia. The overgrowth phenotype results from increased proliferation of undifferentiated hemocytes throughout development and abnormal hemocyte differentiation, suggesting Zfrp8 may participate in cell growth. As described in this example, the role of PDCD2 in human cancer cell growth has now been examined. A survey of several cancer cell lines indicates that PDCD2 is highly expressed in proliferating cells as well as in clinical isolates obtained from patients with hematologic malignancies. PDCD2 knockdown in cancer cells attenuates their proliferation, but not viability relative to parental cells, supporting the notion that PDCD2 over expression facilitates cancer cell growth. Prospective analysis of PDCD2 expression in acute leukemia patients indicates near universal high PDCD2 expression at diagnosis that falls dramatically as patients respond to therapy suggesting utility of PDCD2 expression as a biomarker to follow in leukemia patients as they undergo treatment. The involvement of PDCD2 in cancer cell proliferation is further described in Barboza et al., “PDCD2 functions in cancer cell proliferation and predicts relapsed leukemia”, Cancer Biology & Therapy, 14:6 546-555 (2013), which is hereby incorporated by reference.

Programmed Cell Death 2 (PDCD2) is an evolutionarily conserved eukaryotic protein with unknown function. The original PDCD2 clone (RP-8) was isolated as a death-associated cDNA from a subtractive library enriched for sequences expressed in dexamethasone-treated primary rat thymocytes (Owens et al., (1991) Mol Cell Biol 11:4177). In support of PDCD2 involvement in cell death, enforced expression of PDCD2 in human cell lines has been associated with a modest increase in apoptosis. However, PDCD2 involvement in apoptosis remains unclear, as other studies including genetic analysis in Drosophila failed to uncover additional evidence for PDCD2 apopototic function.

The Drosophila PDCD2 ortholog Zinc finger protein RP-8 (Zfrp8) has an essential function in fly hematopoiesis (Minakhina et al., (2007) Development 134:2387). In mice, PDCD2 also plays an apparent essential role in cell growth and development. However, the biological function of PDCD2 in humans remains unknown. Biochemically, PDCD2 has been shown to physically interact in a yeast two-hybrid assay with host cell factor 1 (HCF-1), a protein whose function is required for cell cycle progression and contributes to the activation of E2F-responsive promoters via its association with mixed-lineage leukemia (MLL) and Set-1 histone H3 lysine 4 methyltransferases. Interestingly, both proteins are targets of ubiquitination, a common strategy to finely regulate the levels of proteins that govern cell cycle progression.

As described herein, the role of PDCD2 in human cancer cell growth has been investigated. It has been shown that human PDCD2 can rescue the lethality and altered hematopoiesis associated with Zfrp8 mutation in Drosophila, confirming the evolutionary conservation of PDCD2 function. PDCD2 is highly expressed in autonomous human cancer cell lines. Moreover, similar high PDCD2 expression was present in clinical isolates obtained from patients with acute leukemia. Prospective analysis of PDCD2 expression in acute leukemia patients indicates near universal high PDCD2 expression at diagnosis that falls dramatically as patients respond to therapy, suggesting the utility of PDCD2 expression as a clinical biomarker to follow in leukemia patients as they undergo treatment. PDCD2 knockdown in two different cancer cell lines (Jurkat leukemia cells and A549 lung carcinoma cells) attenuates their proliferation, but not viability relative to parental cells, supporting the notion that PDCD2 over-expression facilitates cancer cell growth. PDCD2 protein expression is associated with cell proliferation with PDCD2 levels being highest in actively growing cultures, then declining with increasing cell density and increasing contact inhibition. In sum, a novel function for PDCD2 as a facilitator of cancer cell growth has been identified, which suggests this evolutionarily conserved protein may represent a new molecular target for cancer therapies. Also, evidence that PDCD2 expression may serve as correlate for disease status to monitor in acute leukemia patients has been provided.

### Materials and Methods

### PDCD2 and Zfrp8 Sequences

PDCD2 sequence information may be found at GenBank Accession No. NM_{—}002598.3. Zfrp8 sequence information may be found at GenBank Accession No. NM_{—}138046.1.

### Drosophila Strains and Phenotypic Rescue Experiments

The N-terminally GFP-tagged (UAS-GFP-Zfrp8 and UAS-GFP-PDCD2) constructs were made by RT-PCR of the Drosophila Zfrp8 and human PDCD2 coding regions. Both genes were cloned into the Gateway vector pDONR4 (Life Technologies) and subsequently transferred into pPGW (Gateway, Carnegie Institution). Several transgenic fly lines were created following standard protocols (Rubin G M, Spradling A C (1982) Science 218:348; Brand A H, Perrimon N (1993) Development 118:401). Transgene expression was tested using gene-specific and anti-GFP antibodies (data not shown).

T(2;3)B3, CyO; TM6B, Tb¹ balancer used to identify mutant flies and the da-GAL4 driver were obtained from the Bloomington Stock Center (Bloomington, Ind.). Df(2R)SM206 is used as Zfrp8^{null} allele (Minakhina et al., (2007) Development 134:2387).

### Immunostaining

For lymph gland analysis, third instar larvae (24 h before pupation) of each genotype were dissected in phosphate-buffered saline (PBS). Lymph glands with adjacent pericardial cells, brain and discs were immediately fixed for 30 minutes in 4% formaldehyde in PBST (PBS, 0.1% Tween 20) and washed several times in PBST. Antibody staining was performed as described (Minakhina et al., (2007) Development 134:2387; Jung et al., (2005) Development 132:2521). Between 20 and 40 larvae of each genotype were stained with each antibody. Antibodies specific for plasmatocytes (P1) were obtained from Dr I. Ando (Biological Research Center, Szeged, Hungary) and used at 1:400 dilution. Rabbit anti-PPO2 antibody was obtained from George Christophides (Imperial College, London, 1:2000). Secondary goat anti-mouse Cy3 and goat anti-rabbit Cy5 (Jackson ImmunoResearch Laboratories, West Grove, Pa.) were used at 1:500. DNA was stained using Hoechst 33258 (Molecular Probes, Eugene, Oreg.). Samples were mounted in Vectashield (Vector Laboratories, Burlingame, Calif.). Lymph gland images were captured using a Leica DM IRBE laser scanning confocal microscope. The images were analyzed with Image Pro Plus and Leica Microsystems software and further processed using Adobe PhotoShop.

### Quantitative Real Time RT-PCR Analysis

Acute leukemia patients were enrolled in a clinical trial to prospectively measure PDCD2 expression. All patients provided informed assent/consent approved by the Robert Wood Johnson Medical School Institutional Review Board. Peripheral blood and/or bone marrow samples were obtained at diagnosis of acute leukemia and at complete remission, indicated by recovery of peripheral blood counts [absolute neutrophil count (ANC)≧1000; platelet count≧100,000] and no morphological evidence of persistent leukemia, or once best hematopoietic recovery was achieved in cases of refractory disease. The clinical characteristics of the 34 patients analyzed by Quantitative real time RT-PCR (qRT-PCR) is presented in Table 1. All patient peripheral blood and/or bone marrow samples were subjected to red blood cell lysis and if adequate cell numbers were present based on the complete blood cell count; samples were further purified by histoplaque centrifugation (Sigma-Aldrich; catalogue number 10771) using standard methodologies. Total RNA was initially isolated from patient samples using Trizol Reagent (Invitrogen Corporation; catalogue number 15596-026), according to the manufacturer's instructions. To remove potential genomic DNA contamination, RNA was treated with RNase-free DNase Turbo DNA-free kit (Ambion/Applied Biosystems; catalogue number AM1907) prior to amplification. DNase-treated total RNA was isolated from later samples using the RNeasy Mini Kit (Qiagen, Inc.; catalogue number 74104). Quantitative Real-Time RT-PCR was performed using the AgPath-ID One-Step RT-PCR Kit (Ambion/Applied Biosystems; catalogue number AM1005) as per manufacturer's recommendations. The β-actin levels were measured using the following primers: Left primer 5′-GTCTTCCCCTCCATCGTG-3′(SEQ ID NO:1); Right primer 5′-CATGTCGTCCCAGTTGGTG-3′(SEQ ID NO:2); probe (5′6-FAM) CATCCTCACCCTGAAGTACC-(3′ Iowa Black®FQ)(Integrated DNA Technologies) (SEQ ID NO:3). The PDCD2 primers used are as follows (5′ to 3′): Left GCTGCATCTTCCTCTTCTGC (SEQ ID NO:4); Right GGGGAGGGATTCTCAGAAGGT (SEQ ID NO:5); and probe (5′-6-FAM) CCTGCGAGTTTTTAGGAATCA (3′Iowa Black® FQ) (Integrated DNA Technologies) (SEQ ID NO:6). Real-time PCR data was acquired on a Stratagene Mx4000 or a Mx3005P instrument (Agilent Technologies). Samples were analyzed in triplicate. The level of PDCD2 RNA, normalized to the housekeeping gene β-actin, was compared to PDCD2 transcripts levels in normal bone marrow (STEMCELL Technologies Inc.; catalogue number ABM008F) using the comparative C(T) method (Schmittgen T D, Livak K J. Nat Protoc 2008; 3:1101-8).

### Human Hematopoietic Progenitor Cell Culture

Human CD34⁺ bone marrow cells were cultured in methylcellulose according to manufacturer's recommendations (STEMCELL Technologies Inc.; catalogue number ABM017F). To insure an appropriate number of hematopoietic colonies per plate, varying cell numbers ranging from 0.25-1×10⁴ cells were added to 3 ml of complete Methocult® medium (STEMCELL Technologies Inc.; catalogue number H4434) per 35 mm culture dish. After a 10 day period, the Colony-Forming Unit-Erythroid (CFU-E), Granulocyte, Macrophage (CFU-GM) and Granulocyte, Erythrocyte, Macrophage, Megakaryocyte (CFU-GEMM) colonies were identified based on morphology in a dissecting microscope and harvested. Multiple colonies of the same lineage were pooled in IMDM with 2% FBS and processed for protein isolation.

### Cell Lines

The A549 lung carcinoma cell line was obtained from the American Type Culture Collection and maintained in Dulbecco's modified Eagle's (DMEM)/F-12 medium supplemented with 10% heat inactivated fetal bovine serum (FBS) plus 2 mM glutamine, penicillin 100 U/mL and streptomycin 100 U/mL (Invitrogen Corporation). Jurkat T cells were maintained in RPMI medium with the same supplements. Cells were incubated at 37° C. with 5% CO₂.

### PDCD2 Knockdown Constructs and Transfection

A panel of five Dicer Substrate RNAi duplexes were identified using the IDT SciTools RNAi Design algorithm (Integrated DNA Technologies), two of which corresponding to human PDCD2 nucleotides 738-762 (siRNA2) and nucleotides 768-792 (siRNA1) (GeneBank accession number NM_{—}002598.2) yielded the greatest knockdown of PDCD2 protein expression in transient transfection assays and were used for subsequent experiments. Stable PDCD2 knockdown was achieved using a retrovirus-based mammalian RNAi expression system (pSuper RNAi System, OligoEngine; catalogue number VEC-pRT-006). Complementary sense and antisense oligonucleotide sequence corresponding to nucleotides 738-762 (siRNA2) of human PDCD2 was synthesized, annealed and then ligated into pSuper (pSUPER-shPDCD2). A PDCD2 cDNA resistant to siRNA2 was constructed by changing the nucleotide sequence of the siRNA2 interacting site in PDCD2 while maintaining the PDCD2 amino acid sequence. The human PDCD2 coding sequence was mutated to add a unique BamH1 site at nucleotide 760 at the 3′ end of the siRNA2 interacting site. This was accomplished by site-directed mutagenesis (QuikChange II-E-Site Directed Mutagenesis Kit; Agilent Technologies; catalogue number 200555) using the following oligonucleotides (5′ to 3′): PDCD2 Bam sense-GAAGATTACTCAGAGATTATAGGATC CATGGGTGAAGCACTTGAGG (SEQ ID NO:7); PDCD2 Bam reverse-CCTCAAGTGCTTCACCC ATGGATCCTATAATCTCTGAGTAATCTTC (SEQ ID NO:8). The remaining siRNA2 PDCD2 interacting site was mutated using the following oligonucleotides (5′ to 3′): siRNA-resistant sense oligo: TGAGGTTGTGGAAAAAGAGGACTATAGTGAAATCATCG (SEQ ID NO:9); shRNA-resistant anti-sense oligo: GATCCGATGATTTCACTATAGTCCTCTTTTTCCACAACC (SEQ ID NO:10). These oligos were annealed and then ligated into the PDCD2-BamH1 construct restricted with Bsu36I and BamH1 generating the siRNA2-resistant PDCD2 construct where 12/25 nucleotides of the siRNA2 interacting site are mutated while the PDCD2 amino acid sequence is maintained. The resultant siRNA-resistant PDCD2 cDNA was amplified by PCR adding a 5′ Bg1II site and a 3′ XhoI site to facilitate cloning into the Murine Stem Cell Virus vector pMSCVpuro (Clontech; catalogue number 6311461) yielding the pMSCV-huPDCD2-shRNA^{R} construct.

Cells were transiently transfected with Dicer Substrate RNAi duplexes (Integrated DNA Technologies) using Lipofectamine 2000 (Invitrogen Corporation; catalogue number 11668-019) following manufacturer's recommendations. A549 cells were seeded at a density of 0.2×10⁶ cells per well in a 6 well plate, then 24 hrs later transfected with Dicer Substrate RNAi duplexes (final concentration 10 nM), The transfection was repeated 24 hours later with a lower concentration of Dicer Substrate RNAi duplexes (5 nM). Transfected cells were split one day after the final transfection and cultured for 5 additional days. At the end of the 5 day period, cells were harvested for automated trypan blue exclusion using a Vi-Cell™ Cell Viability Analyzer (Beckman-Coulter), and Western analysis. Each transfection was performed in triplicate and the overall transfection experiment was repeated at least twice. The siRNA duplexes for human PDCD2 are as follows: siRNA1, 5′-CAGTTCTTCCTCAAGTGCTTCACCCAT-3′ (SEQ ID NO:11), siRNA2, 5′-CCCTATAATCTCTGAGTAATCTTCCTT-3′ (SEQ ID NO:12).

A549 cells were stably transfected with the pSuper-shPDCD2 construct using Lipofectamine 2000 as per manufacturer's recommendations. Stable G418^{R} colonies were selected and screened for PDCD2 knockdown by Western blotting. For Jurkat cells, cells were transfected with pSuper-shPDCD2 using the Amaxa Nucleofector II program X-001, then subjected to G418 selection and cloned by limiting dilution. The clonal Jurkat cell lines were screened for PDCD2 knockdown (KD) by Western analysis. Jurkat PDCD2 KD cells were maintained at a density of 1×10⁵ cells/ml and the A549 PDCD2 KD at a maximum confluence of 25%.

Retroviral particles were produced by transient transfection of pMSCV-huPDCD2-shRNA^{R} into the Phoenix packaging cell line as described previously (Pear, et al., Proc Natl Acad Sci USA 1993; 90:8392-6). Media conditioned by the transfected Phoenix packaging cells was used to infect A549 and A549-PDCD2 KD cells in the presence of 4 μg/ml polybrene. Infected cultures were maintained in 1.5 μg/ml puromycin. After selection, 0.25×10⁶ puromycin^{R} cells were plated per well of 6-well plates and allowed to grow. Cells were harvested 72 hours later for cell counts and western analysis. Triplicate wells were counted using a Vi-Cell™ Cell Viability Analyzer (Beckman-Coulter).

### Cell Cycle Analysis

EdU (5-ethynyl-2′-deoxyuridine; Invitrogen Corporation; catalogue number C10424) incorporation was used to measure G1/S progression. Relevant cell lines were seeded in triplicate at 0.5×10⁶ cells per 10 cm plate then 72 hours later were pulsed for 2 hours with EdU (10 μM). Cells were harvested, fixed and processed for EdU incorporation by Alexa Fluor® 647 detection with flow cytometry according to the manufacturer's protocols. For cell proliferation, the percentage of newly synthesized DNA was determined as percentage of Alexa Fluor® 647 stained cells using the CellQuest software. A minimum of 10,000 cells per sample was analyzed. The results presented are representative of at least three experiments. Cell cycle analysis was performed using standard methodologies [Propidium Iodide (PI) RNase staining buffer, BD Pharmingen; catalogue number 550825]. Fluorescent intensities were acquired on a BD FACSCalibur™ and the percentage of cells in each phase of the cell cycle was calculated using the ModFit v3.0 software (BD Biosciences).

### Protein Expression

Western analysis was done using standard methods. Polyclonal antibody against PDCD2 was obtained from the Sharp Laboratory (Scarr R B, Sharp P A. Oncogene 2002; 21:5245-54), mouse monoclonal Rb (556538) and p27 (554069) were purchased from BD Biosciences; Cdk4 (sc-601), Cdk6 (sc-177) and Cdc2 (sc-54) antibodies purchased from Santa Cruz Biotechnology, Inc. Monoclonal antibody against β-actin (A1978) was purchased from Sigma-Aldrich and anti-glyceraldehyde-3-phosphate dehydrogenase-horseradish peroxidase (HRP) conjugate (GAPDH; 3683) was purchased from Cell Signaling, Inc.

### Statistical Analysis.

Statistical analyses were carried out using the two tailed Student's t-test. A p value less than 0.05 was considered to be statistically significant. All statistical analyses were conducted using the GraphPad Prism® software program (version 5, Graphpad Software, Inc.).

### Results

### The Human Homolog, PDCD2, Rescues the Zfrp8 Phenotype

In Drosophila Zfrp8 mutants were identified based on their severe abnormalities of the hematopoietic organ, the lymph gland. Zfrp8 mutations are lethal before adulthood and larvae show strongly overgrown lymph glands, up to 30 fold, and abnormalities in hemocyte differentiation. The Zfrp8 mutant lymph glands show premature prohemocyte differentiation in the medullary zone into immature hemocytes that ultimately differentiate into the three mature blood cell types (plasmatocytes, crystal cells and lamellocytes) (Minakhina S, Steward R (2010) Development 137:27). Clonal analysis has shown that the gene is essential in HSCs.

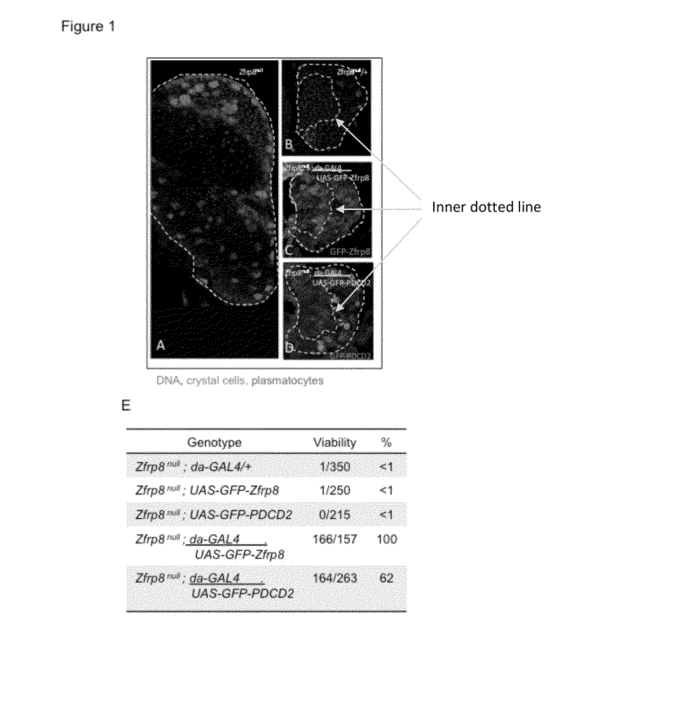

Human PDCD2 and Drosophila Zfrp8 amino acid sequences are 40% identical and >50% similar over their entire length. It has been previously shown that general expression of a Zfrp8 cDNA in mutant animals rescues all aspects of the Zfrp8 null phenotype. To ascertain whether the human gene could substitute for the Drosophila Zfrp8 functions in vivo, transgeneic flies expressing GFP-tagged Zfrp8 and GFP-PDCD2 were created. In all the rescue experiments the lethal Zfrp8 null allele were used as host (FIG. 1B) and the transgenes were expressed under the control of the daughterless general promoter. Flies expressing the Drosophila GFP-Zfrp8 transgene show complete rescue (FIG. 1). Importantly, 62% of Zfrp8 mutant flies expressing the human GFP-PDCD2 transgene survive to adulthood and show normal development, lifespan, and fertility. The appearance of the Zfrp8 and PDCD2-rescued lymph glands is normal, albeit with a slight increase in size, similar to that of Zfrp8 heterozygous animals. The medullary zone, lost in the homozygous mutant, is easily recognized (outlined with the inner dotted line, FIG. 1B-D). The efficient rescue of lethality and of the hematopoietic phenotypes of Zfrp8^{null} animals by human PDCD2 is conclusive evidence of the functional conservation of the human and Drosophila Zfrp8/PDCD2.

### High PDCD2 Expression is Characteristic of Human Hematopoietic Progenitors

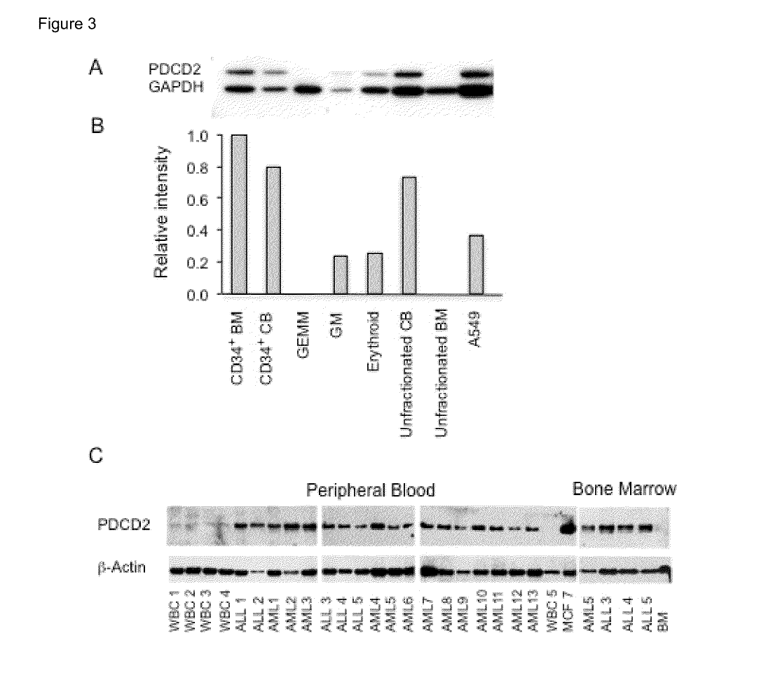

Based on experiments in Drosophila that show Zfrp8/PDCD2 is essential for the maintenance of hematopoietic stem cells (HSCs) (Minakhina S, Steward R. Hematopoietic stem cells in Drosophila. Development; 137:27-31), evidence was sought that PDCD2 is also expressed by human hematopoietic progenitor cells. Using colony formation assays, PDCD2 protein expression was evaluated in hematopoietic progenitors of varying lineage commitment (FIG. 3A, B). PDCD2 expression was highest in the cell fraction enriched for totipotent hematopoietic stem cells, namely the bone marrow CD34+ population, and not detectable in unfractionated bone marrow, where the frequency of CD34+ positive cells is approximately 1 in 10² bone marrow cells (Collins R H, Jr. Stem Cells 1994; 12:577-85). PDCD2 expression was also elevated in human cord blood progenitors, an alternate source of hematopoietic stem cells [recently reviewed in (Mayani H. Arch Med Res 2011; 42:645-51)]. PDCD2 protein levels varied inversely with hematopoietic differentiation with lower PDCD2 expression present in more lineage committed progenitors (GM, erythroid and GEMM colonies).

### PDCD2 is Highly Expressed in Human Cancer

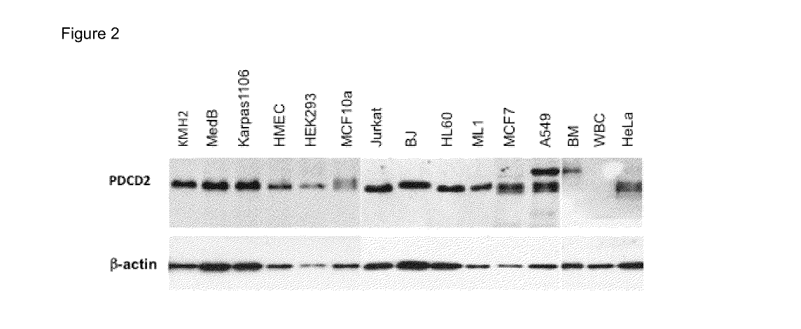

PDCD2 is present in eukaryotes from yeast to humans and mammalian proteins are >80% identical along their entire length. Human PDCD2 maps to chromosomal band 6q27, a region associated with translocations found in leukemia and lymphomas (Kawakami et al., (1995) Cytogenet Cell Genet 71:41; Amiel et al., (1999) Cancer Genet Cytogenet 112:53; Stilgenbauer et al., (1999) Leukemia 13:1331). PDCD2 is a target of BCL-6 (B-Cell Lymphoma Protein 6) and has been shown to have an expression pattern that is inversely related to that of this transcriptional repressor in human B-cell lymphomas (Baron et al., (2002) Proc Natl Acad Sci USA 99:2860; Baron et al., (2007) Proc Natl Acad Sci USA 104:7449). Although PDCD2 was suggested to be a putative tumor suppressor, no systematic investigation of its expression in tumors has been published (Steinemann et al., (2003) Genes Chromosomes Cancer 37:421). To gain insight into PDCD2 function several cancer cell lines were surveyed for PDCD2 protein expression. Western analysis shows that PDCD2 is expressed in a variety of cancer cell lines; including leukemia and carcinoma cells, with all cell lines examined having increased levels of PDCD2 protein when compared to primary peripheral blood and bone marrow mononuclear cells (FIG. 2). Given the association of Zfrp8 mutations with altered Drosophila hematopoiesis, and high levels of PDCD2 expression in cell lines derived from leukemias and lymphomas, (FIG. 2), peripheral blood samples obtained from patients with acute leukemia were analyzed. Patient blood samples that contained ≧70% leukemic blasts, as well as patient bone marrow isolates were evaluated for PDCD2 protein expression. PDCD2 was highly expressed in all human acute leukemia samples examined (FIG. 3C). PDCD2 expression was barely detectable in peripheral blood and bone marrow mononuclear cells obtained from normal controls, again suggesting high PDCD2 protein expression is a characteristic of acute leukemia cells.

### Prospective Analysis of PDCD2 in Acute Leukemia Patients

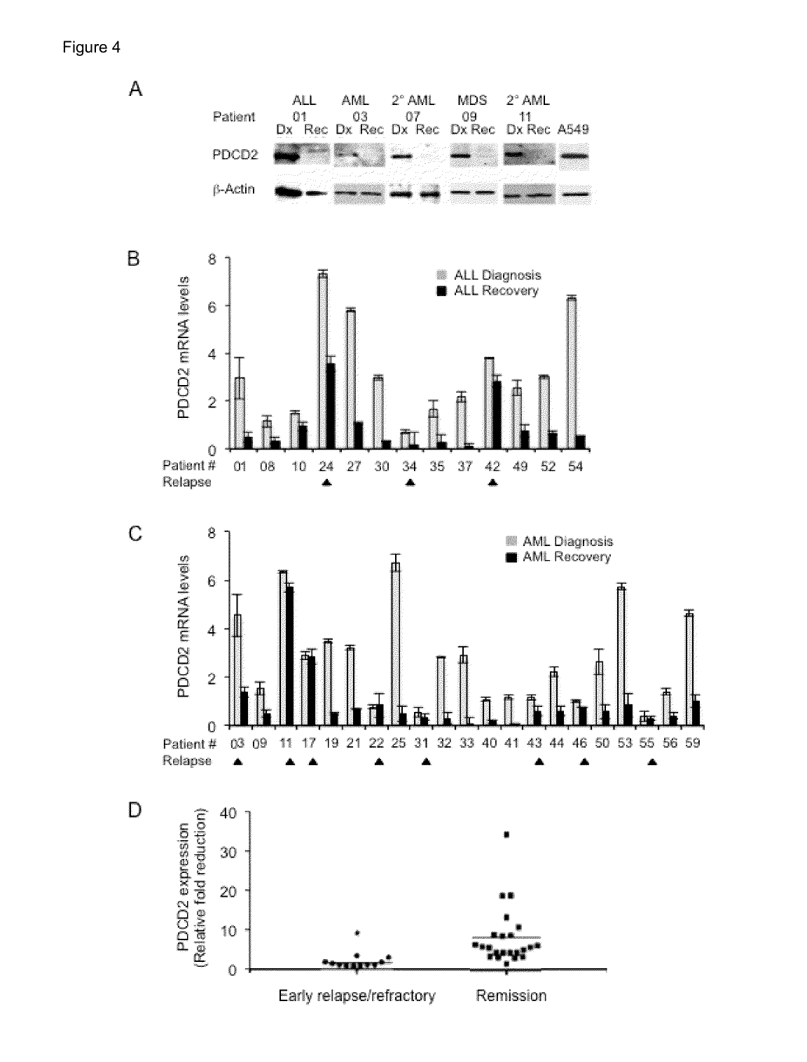

To further implicate PDCD2 in human malignancies, PDCD2 expression was prospectively analyzed in patients with acute leukemia in whom its expression could be followed temporally as patients present and undergo treatment. Peripheral blood and/or bone marrow samples were collected at the time of diagnosis and at complete remission or, in patients with persistent disease, once best hematologic recovery was attained usually approximately Day 30 after induction chemotherapy. Patient bone marrow (BM) samples were initially analyzed by Western analysis with a representative panel of acute leukemia/advanced myelodysplasia patient samples presented in FIG. 4A. At diagnosis PDCD2 protein levels were high, similar to what was found in the initial survey of clinical isolates (FIG. 3C). After treatment PDCD2 protein levels were markedly lower, barely detectable by Western analysis, suggesting PDCD2 protein expression may correlate with disease status.

To more precisely measure PDCD2 expression, a quantitative Real Time RT-PCR (qRT-PCR) assay was developed and utilized to monitor PDCD2 RNA expression in subsequent patients. PDCD2 RNA is expressed at low levels in normal, unfractionated BM, which provided a means to quantitate the elevated PDCD2 RNA expression present in leukemia patient samples. PDCD2 RNA levels were normalized to β-actin message as a loading control and presented relative to the level of PDCD2 RNA in normal total bone marrow (e.g. a value of 1 would indicate equivalent PDCD2 RNA levels to normal bone marrow). PDCD2 transcripts were amplified at diagnosis and at hematopoietic recovery post-induction chemotherapy in thirty-four acute leukemia patients. These qRT-PCR results indicate that PDCD2 RNA is expressed at much higher levels at diagnosis than at hematopoietic recovery post-treatment in the vast majority of patients analyzed (FIG. 4 and Table 1). The qRT-PCR proved to be more sensitive than Western analysis as illustrated by patient 11, where Day 30 PDCD2 protein levels were not detectable (FIG. 4A) due to the paucity of bone marrow cells remaining as a result of treatment, but PDCD2 RNA was still detectable and relatively unchanged by qRT-PCR (FIG. 4C).

In 13 acute lymphoblastic leukemia (ALL) patients, PDCD2 RNA levels are consistently high at diagnosis and markedly decreased after treatment (FIG. 4B). There have been two relapses (patients 24 and 34) and one patient with refractory disease (42) in this group thus far and in ⅔ of these relapsed/refractory cases, PDCD2 RNA levels remained elevated post-treatment (24 and 42). In AML patients, more patients have relapsed, and a trend of PDCD2 RNA expression emerges where the fold reduction in PDCD2 RNA levels appears to be important (FIG. 4C). Thus far 7/20 AML patients have relapsed (03, 17, 22, 31, 43, 46 and 55) and one patient (11) had refractory disease. AML patients that have remained in clinical remission to date exhibit an average 9.34 fold reduction in PDCD2 RNA levels at hematologic recovery after receiving chemotherapy. However, AML patients that relapsed or had refractory disease exhibited only an average reduction in PDCD2 RNA levels of 1.52 fold after treatment. This fold reduction in PDCD2 RNA level does not appear to correlate with a specific cytogenetic abnormality in this small sample of AML patients, with relapses roughly distributed between intermediate and poor prognostic cytogenetic categories. Combining the ALL and AML data, the difference in PDCD2 RNA reduction after treatment in relapsed/refractory patients versus those who maintained a complete remission reached statistical significance. Patients with refractory disease or who relapsed had an average fold reduction of 1.74 after treatment, whereas patients who achieved a complete remission had a much greater average fold reduction in PDCD2 RNA levels of 8.33 (FIG. 4D; p=0.006). These data suggest that PDCD2 RNA expression correlates with disease status and the fold reduction in PDCD2 RNA after initial induction chemotherapy appears to be a significant predictor of relapse in acute leukemia patients.

### PDCD2 Knockdown Impairs Cancer Cell Proliferation

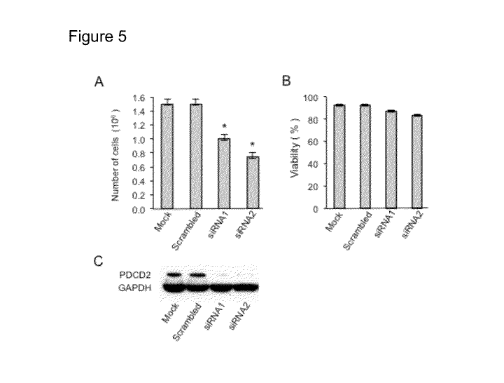

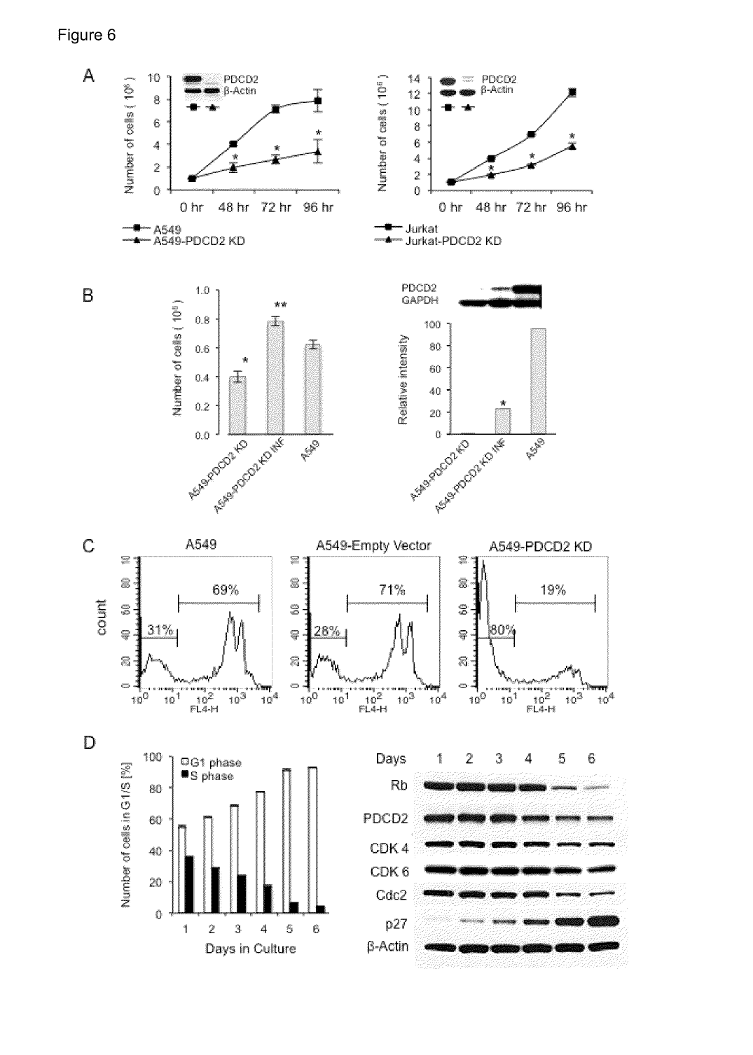

The high PDCD2 expression seen in primary acute leukemia cells suggests this protein may play a role in their malignant properties, such as high rates of cell proliferation. This possibility was investigated by transiently transfecting shRNAs targeting human PDCD2 into the readily transfectable lung carcinoma cell line A549, which has a high basal expression of PDCD2 (FIG. 2). These results indicate PDCD2 knock down by two separate shRNAs targeting different PDCD2 sequences, impaired A549 cell proliferation in a statistically significant fashion (FIG. 5). Interestingly, PDCD2 knock down did not significantly affect cell viability, implying the decreased cell proliferation that resulted from PDCD2 repression is due to the slowing of cell growth rather then to a direct cytotoxic effect. To further analyze PDCD2 function, its protein expression was stably knocked down using a retrovirus-based shRNA expression vector in two cancer cell lines, A549 lung carcinoma cells and Jurkat leukemia cells (FIG. 6A). PDCD2 knockdown (KD) yielded similar phenotypes in both cell lines. PDCD2 KD cells exhibited significantly slower cell growth relative to parental A549 and Jurkat cells as measured by viable cell number. Again PDCD2 expression was not essential for cancer cell viability as PDCD2 KD cell cultures had the same viability over time as their parental counterparts (data not shown). In order to confirm the specificity of the PDCD2 knock down effect on lung cancer cell proliferation, the shRNA interacting site targeted by siRNA2 in PDCD2 was extensively mutated at the nucleotide level while maintaining the correct PDCD2 amino acid sequence using Wobble codons. The shRNA^{R} PDCD2 cDNA was cloned into a murine stem cell virus vector and used to infect the A549 PDCD2 KD cell line that over expresses siRNA2. These results shown in FIG. 6B indicate the shRNA^{R} PDCD2 cDNA partially restored PDCD2 protein expression and rescued the effects of PDCD2 knock down on A549 lung cancer cell proliferation in a statistically significant fashion. In sum these results provide evidence that PDCD2 repression significantly impairs cancer cell growth.

The inhibitory effect of PDCD2 knockdown on cell growth was further investigated in the stable A549 PDCD2 KD lung carcinoma cells. Cell cycle analysis failed to uncover significant differences between PDCD2 KD cell lines and the parental controls (data not shown), however EdU incorporation revealed PDCD2 KD A549 cells exhibited a much lower degree of EdU uptake [19% EdU positive PDCD2 KD cells compared with A549 parental and pSuper-empty vector 69% and 71% EdU positive cells repectively; FIG. 6C] indicating a lower percentage of PDCD2 KD cells passing through S phase. The EdU uptake data, in conjunction with the lack of a specific cell cyle checkpoint block, suggests that PDCD2 knockdown results in an overall slowing of the cell cycle.

These observations suggest that PDCD2 function is associated with cellular proliferation. As a means to investigate this possibility, PDCD2 protein expression was measured as a function of time, cell culture density and G1/S progression. A549 cells (PDCD2 intact) were initially plated at sub-confluency and allowed to reach confluence. Daily timepoints were analyzed for the percentage of cells in G1 or S phase and the expression PDCD2 along with other cell cycle regulators (FIG. 6D). Interestingly, actively dividing cultures, as indicated by the highest percentage of cells in S phase, contained the highest levels of PDCD2. As cultures reached confluency and cell growth slowed, marked by the decline in percentage of cell in S phase and increase in G1 cells, PDCD2 expression decreased precipitously. The cell cycle exit due to increasing cell density was confirmed by the induction of the cyclin dependent kinase inhibitor p27, whose expression was inversely related to PDCD2 expression. PDCD2 shared a similar expression pattern with other proteins involved with cell division, cyclin dependent kinase 1 (Cdc2), cyclin dependent kinase 4 (Cdk4), cyclin dependent kinase 6 (Cdk6) and retinoblastoma protein (Rb), whose expression also decreased with increasing cell density and subsequent cell cycle exit. These results indicate that PDCD2 likely participates in cell growth and its high degree of expression in cancer cells may contribute to the uncontrolled growth characteristic of malignant cells.

Multiple lines of evidence suggest that PDCD2 may play an important role in human oncogenesis. Zfrp8 mutations in Drosophila cause an enormous overgrowth of the lymph gland, the primary site of fly hematopoiesis. This overgrowth phenotype, although suggestive of Zfrp8 acting as a tumor suppressor, was found to be the result of changed cellular homeostasis and a delay in prohemocyte differentiation. Genetic analyses have revealed the loss of Zfrp8 affected the stem and precursor cells in the lymph gland without changing the fate of pluripotent progenitors and cells with limited mitotic potential (Minakhina S, Steward R. Hematopoietic stem cells in Drosophila. Development; 137:27-31). These results indicate that Zfrp8 has a stem cell function and its loss affects HSC self-renewal and not the differentiation of more committed hematopoietic progenitors. The results presented here suggest that PDCD2 may have a similar HSC-specific function in human hematopoiesis. PDCD2 expression is high in HSC-enriched populations (cord blood and CD34⁺ bone marrow cells) and diminishes with further differentiation. Likewise PDCD2 over expression in acute leukemia cells is likely a reflection of the primitive nature of this malignant population.

This clinical study of acute leukemia patients, showed that the fold reduction PDCD2 RNA levels after initial treatment may be a predictor of relapse in these patients. Specifically, patients with persistent levels of PDCD2 expression were statistically more likely to relapse than those exhibiting marked decreases in expression. Moreover, the high level of PDCD2 expression in leukemia cells is reminiscent of PDCD2 expression in primitive hematopoietic progenitors, and may be reflective of the proliferative potential of these cellular populations relative to normal, differentiated hematopoietic cells. These observations suggest PDCD2 or it's over expression, may play an active role in leukemogenesis and represent a new therapeutic target for acute leukemia. Patients who relapsed or had refractory disease had a much lower fold reduction in PDCD2 RNA levels (mean ratio=1.74); while patients in complete remission had a much higher fold reduction PDCD2 RNA levels after induction chemotherapy (mean ratio=8.33). This relationship between relapse and the ratio of before- and after-treatment PDCD2 levels will require validation in prospective analysis of a larger panel of acute leukemia patients. PDCD2 expression levels may provide a means to not only identify patients early in the course of treatment who are at higher risk for relapse, but also may be a biomarker that can be followed in surveillance for early relapse once a complete remission is obtained for both ALL and AML patients.

There is a clear need to identify additional biomarkers to better manage patients with AML. In particular, patients with normal cytogenetics comprise the largest subgroup and have very disparate outcomes. A significant percentage will be cured with standard chemotherapy, perhaps 30%, however the majority will eventually relapse and require a different treatment modality, such as allogeneic stem cell transplantation. Without cytogenetic abnormalities, it is not possible to accurately predict at diagnosis which AML patients will relapse, therefore it is imperative to identify new molecular markers to better stratify patients by relapse risk and plan treatment accordingly. Likewise it would be beneficial to identify patients with refractory disease, e.g. those destined to relapse within six months of induction chemotherapy. These patients with refractory disease would be spared additional toxicity of receiving intensive chemotherapy with a low likelihood of inducing a meaningful period of complete remission. Thus, the utility of a sensitive quantitative PCR assay for PDCD2 RNA for its prognostic implications and as an early biomarker of AML relapse is of potential therapeutic importance.

The presence of discrete molecular lesions has allowed the development of PCR assays that more precisely measure minimal residual disease in select hematologic malignancies. In myeloid leukemias, the Philadelphia chromosome t(9;22) in chronic myelogenous leukemia and the t(15;17) in acute promyelocytic leukemia thus far have been validated as molecular markers useful in monitoring the care of these patients. In addition, mutations in nucleophosmin 1 (NPM1), FLT3 and CCAAT/enhancer binding protein α (CEBPA α) have been shown to have clinically significant prognostic value. A potential drawback to following these mutations is they are also sometimes lost upon relapse in leukemia blasts, as an example a recent study has shown that the FLT3 mutation status changed in 22% of relapsed patients, including the loss of the FLT3 kinase domain mutation in approximately 10% of patients. Measuring changes in PDCD2 expression by PCR may represent a sensitive, novel approach to monitoring minimal residual disease and/or allow earlier detection of disease relapse in AML patients. This approach provides a means to circumvent the potential loss of mutation/marker that can occur in relapsed disease as PDCD2 is normally expressed BM and is indispensable for normal development based on published Drosophila and mouse studies. The observations described herein suggest that the amount of PDCD2 expression after chemotherapy relative to normal BM, will be predictive of future relapse. Finally, PDCD2 coding and regulatory regions may harbor mutations that alter its normal expression and function that are responsible for its high expression in cancer cells. Although PDCD2 mutations have yet to be reported, sequence analysis of AML clinical samples may reveal somatic PDCD2 mutations similar to other recurrent AML mutations such as those in NPM1 and CEBPA α and provide new insight into leukemogenesis.

Consistent with the high level of PDCD2 expression characteristic of acute leukemia clinical isolates, a new biological PDCD2 function as a facilitator of cancer cell growth was identified. PDCD2 knockdown significantly impairs cancer cell proliferation and the proportion of cells progressing through S phase. The PDCD2 protein expression pattern is similar to other cell cycle regulators that function in G1/S transition, suggesting that PDCD2 may also function in the cellular decision to enter the cell cycle or affect the efficiency the G1/S transition. Additional work is required to ascertain if PDCD2 functions to facilitate cell cycle progression at the transcriptional level, perhaps as a binding factor for other transcriptional regulators such as HCF-1, or via other as yet undescribed protein-protein interactions.

In sum, as described herein, it has been shown that PDCD2 is a marker for normal early hematopoietic progenitor cells and PDCD2 also participates in cancer cell proliferation, consistent with its high level of expression in clinical samples obtained from patients with cancer. Moreover, evidence has been provided that PDCD2 expression, based on its ability to predict poor outcomes, may be exploited as a biomarker to aid surveillance for recurrent disease in acute leukemia patients. PDCD2's role in cell proliferation and it's high expression in human malignancies make it an attractive, novel molecular target for new anti-cancer therapies.

### Example 2

### A New Stem Cell Gene

### Zfrp8/PDCD2

The lymph gland is the hematopoietic organ of Drosophila. Drosophila hematopoiesis shows many similarities to human hematopoiesis and virtually all genes and pathways functioning in Drosophila blood formation also have an essential role in vertebrate hematopoiesis. It has been shown that the embryonic and first instar larval lymph gland contains a few cells that have similar characteristics as vertebrate hematopoietic stem cells (HSC). As discussed above in Example 1, it has been found that the highly conserved Zfrp8/PDCD2 gene has an essential function in HSCs, but is dispensable in more mature cells. The function of Zfrp8 in other stem cells has now been studied and it has been found that it is also required in both germline and somatic stem cells in the Drosophila ovary; mutant stem cells loose their stem cell identity and give rise to abnormal daughter cells that ultimately stop dividing. Further characterization of the role of Zfrp8/PDCD2 in ovarian stem cells is described in Minakhina et al., “Zfrp8/PDCD2 is required in ovarian stem ce3lls and interacts with the piRNA pathway machinery”, Development, 141, 259-268 (2014), which is hereby incorporated by reference. Consistent with these findings, the vertebrate homolog PDCD2 has also been shown to be essential for the viability of mouse embryonic stem cells (Mu et al., (2010) Dev Biol. 2010 Nov. 15; 347(2):279-88). These studies indicate that PDCD2/Zfrp8 has a conserved role in many if not all stem cells of different species.

Accordingly, altered expression of PDCD2 may lead to the dysregulation of germline and somatic stem cells, which ultimately may result in the development of cancer.

### Example 3

### PDCD2 Over-Expression in Human Lung Cancer Tissue Arrays

### Tissue Array Immunohistochemistry Analysis

Lung tissue microarray slide containing 200 cores of a variety of normal lung tissue and lung carcinoma was obtained from US Biomax, Incorporated (Rockville, Md.; catalogue number BCO41114). The slide was stained with PDCD2 immunohistochemistry. The localization and intensity of the staining were evaluated by a pathologist and the results were evaluated by an independent biostatistician, using standards and techniques known in the art.

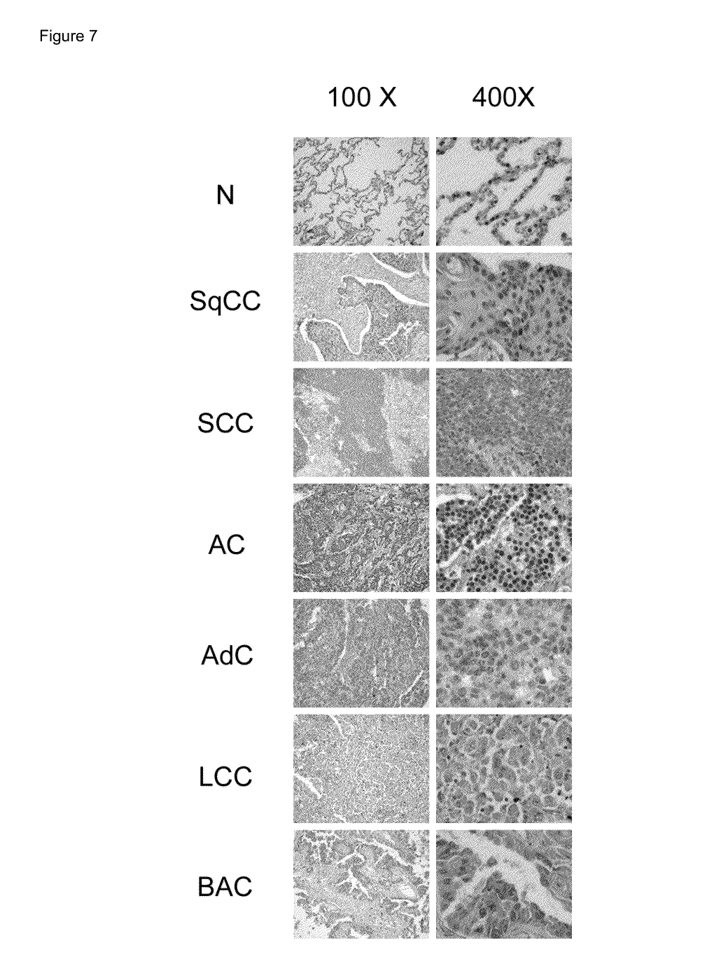

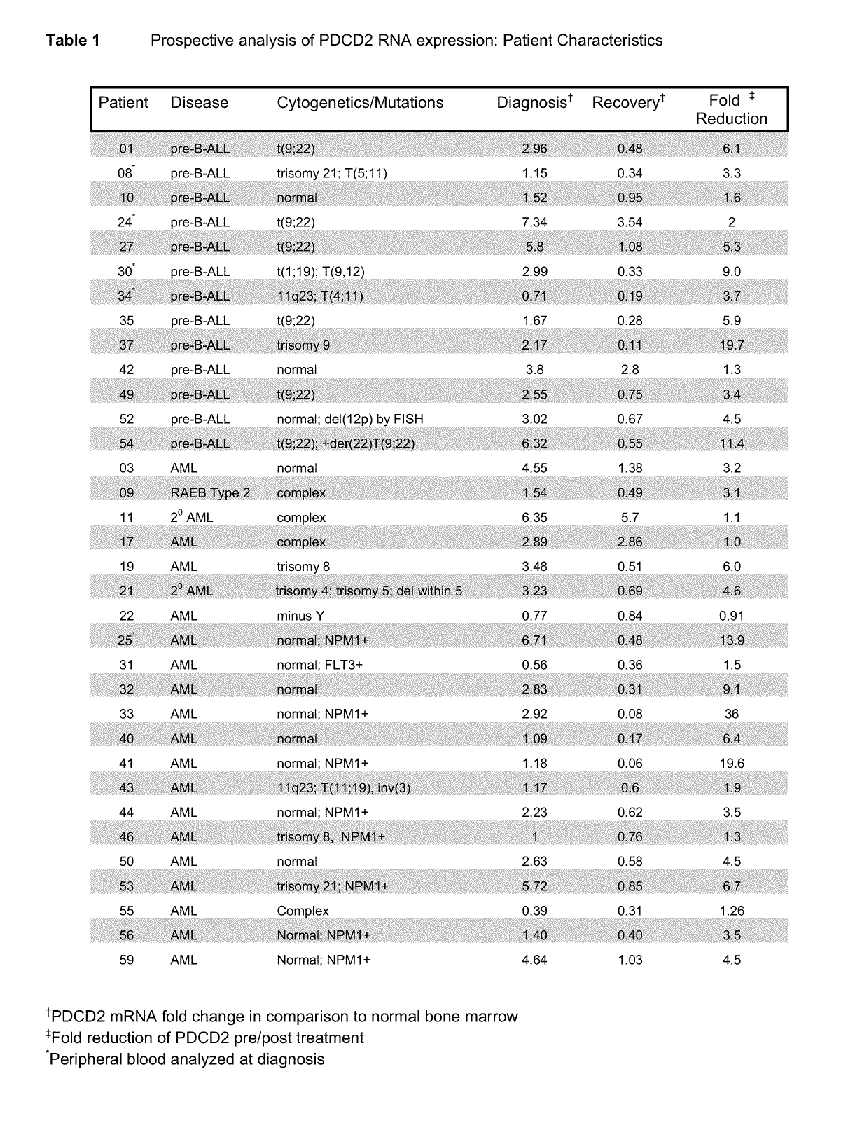

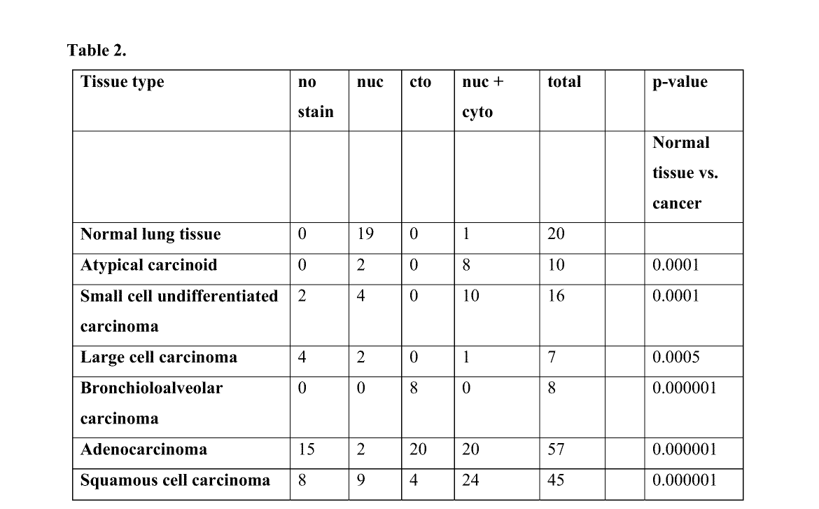

This survey of varied human cancer cell lines indicated that high PDCD2 protein expression was characteristic of human cancer cells with some of the highest levels present in the lung carcinoma cell A549 (FIG. 2). It was next sought if high PDCD2 expression was also a characteristic of primary human lung cancer using tissue arrays. These results are presented in FIG. 7. In normal lung tissue controls, PDCD2 expression is restricted to type II pneumocytes, which have long been considered pulmonary epithelial stem cells that proliferate in response to lung injury [Bishop, A. E. Pulmonary epithelial stem cells. Cell Prolif, 37: 89-96, 2004.] The high PDCD2 expression present in type II pneumocytes is reminiscent of the PDCD2 expression in hematopoietic progenitors (FIG. 3A and B) suggesting PDCD2 may have stem cell-specific function and potentially analogous to Zfpr8 essential function in Drosophila hematopoietic stem cells. PDCD2 expression was also predominately nuclear in the normal lung tissue controls. Analysis of tissue sections derived from six different lung cancer subtypes (squamous cell, small cell, large cell, adenocarcinoma, carcinoid and bronchioloalveolar carcinoma) revealed high PDCD2 expression in most cases. While PDCD2 expression was predominately nuclear in normal lung tissues; a significant degree of cytoplasmic staining was apparent in cancer tissues. The amount of PDCD2 staining was evaluated by a pathologist and the differences in PDCD2 subcellular localization between normal controls and lung cancer samples proved to be highly statistically significant (Table 2). These results indicate that PDCD2 is a predominately nuclear protein that is highly expressed in lung cancer and the loss of PDCD2 nuclear localization appears to be a characteristic of cancer cells.

### PDCD2 Knockdown Impairs Lung Cancer Cell Proliferation.

The high PDCD2 expression seen in primary acute leukemia cells and human lung cancer tissue arrays suggests this protein may play a role in their malignant properties, such as high rates of cell proliferation. This possibility was investigated by transiently transfecting shRNAs targeting human PDCD2 into the readily transfectable lung carcinoma cell line A549, which has a high basal expression of PDCD2 (FIG. 5). These results indicate PDCD2 knock down by two separate shRNAs targeting different PDCD2 sequences, impaired A549 cell proliferation in a statistically significant fashion (FIG. 5). Interestingly, PDCD2 knock down did not significantly affect cell viability, implying the decreased cell proliferation that resulted from PDCD2 repression is due to the slowing of cell growth rather then to a direct cytotoxic effect. To further analyze PDCD2 function, its protein expression was stably knocked down using a retrovirus-based shRNA expression vector in two cancer cell lines, A549 lung carcinoma cells and Jurkat leukemia cells (FIG. 6A). PDCD2 knockdown (KD) yielded similar phenotypes in both cell lines. PDCD2 KD cells exhibited significantly slower cell growth relative to parental A549 and Jurkat cells as measured by viable cell number. Again PDCD2 expression was not essential for cancer cell viability as PDCD2 KD cell cultures had the same viability over time as their parental counterparts (data not shown). In order to confirm the specificity of the PDCD2 knock down effect on lung cancer cell proliferation, the shRNA interacting site targeted by siRNA2 in PDCD2 was extensively mutated at the nucleotide level while maintaining the correct PDCD2 amino acid sequence using Wobble codons. The shRNA^{R} PDCD2 cDNA was cloned into a murine stem cell virus vector and used to infect the A549 PDCD2 KD cell line that over expresses siRNA2. These results shown in FIG. 6B indicate the shRNA^{R} PDCD2 cDNA partially restored PDCD2 protein expression and rescued the effects of PDCD2 knock down on A549 lung cancer cell proliferation in a statistically significant fashion. In sum these results provide evidence that PDCD2 repression significantly impairs lung cancer cell growth.

The inhibitory effect of PDCD2 knockdown on cell growth was further investigated in the stable A549 PDCD2 KD lung carcinoma cells. Cell cycle analysis failed to uncover significant differences between PDCD2 KD cell lines and the parental controls (data not shown), however EdU incorporation revealed PDCD2 KD A549 cells exhibited a much lower degree of EdU uptake [19% EdU positive PDCD2 KD cells compared with A549 parental and pSuper-empty vector 69% and 71% EdU positive cells repectively; FIG. 6C] indicating a lower percentage of PDCD2 KD cells passing through S phase. The EdU uptake data, in conjunction with the lack of a specific cell cyle checkpoint block, suggests that PDCD2 knockdown results in an overall slowing of the cell cycle.

These observations suggest that PDCD2 function is associated with cellular proliferation. As a means to investigate this possibility, PDCD2 protein expression was measured as a function of time, cell culture density and G1/S progression. A549 cells (PDCD2 intact) were initially plated at sub-confluency and allowed to reach confluence. Daily timepoints were analyzed for the percentage of cells in G1 or S phase and the expression PDCD2 along with other cell cycle regulators (FIG. 6D). Interestingly, actively dividing cultures, as indicated by the highest percentage of cells in S phase, contained the highest levels of PDCD2. As cultures reached confluency and cell growth slowed, marked by the decline in percentage of cell in S phase and increase in G1 cells, PDCD2 expression decreased precipitously. The cell cycle exit due to increasing cell density was confirmed by the induction of the cyclin dependent kinase inhibitor p27, whose expression was inversely related to PDCD2 expression. PDCD2 shared a similar expression pattern with other proteins involved with cell division, cyclin dependent kinase 1 (Cdc2), cyclin dependent kinase 4 (Cdk4), cyclin dependent kinase 6 (Cdk6) and retinoblastoma protein (Rb), whose expression also decreased with increasing cell density and subsequent cell cycle exit. These results indicate that PDCD2 likely participates in cell growth and its high degree of expression in cancer cells may contribute to the uncontrolled growth characteristic of malignant cells.

Lung cancer remains the number one cause of cancer mortality in men and women. PDCD2 over expression in lung cancer may provide an additional needed molecular therapeutic target for this disease, akin to the observations of epidermal growth factor receptor (EGFR) and vascular endothelial growth factor over expression giving rise to an array of drugs targeting these signaling pathways. These observations made in lung carcinoma cells indicate repressing PDCD2 expression directly impairs cancer cell growth, this observation coupled with the high PDCD2 protein expression characteristic of both solid tumor and leukemia cells implicate PDCD2 as an exciting new potential target for cancer therapies.

All documents cited herein are incorporated by reference. While certain embodiments of invention are described, and many details have been set forth for purposes of illustration, certain of the details can be varied without departing from the basic principles of the invention.

The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. In addition to the order detailed herein, the methods described herein can be performed in any suitable order unless otherwise indicated herein or otherwise clearly contradicted by context. The use of any and all examples, or exemplary language (e.g., “such as”) provided herein, is intended merely to better illuminate embodiments of invention and does not necessarily impose a limitation on the scope of the invention unless otherwise specifically recited in the claims. No language in the specification should be construed as indicating that any non-claimed element is essential to the practice of the invention.

What is claimed is:

1. A method for identifying a subject that has cancer, or is at an elevated risk for developing cancer, comprising:
a) quantifying the level of Programmed Cell Death 2 (PDCD2) protein or RNA in a biological sample from the subject and comparing the level to a control sample; and
b) determining that the subject has cancer, or is at an elevated risk for developing cancer, based on the level of PDCD2 protein or RNA in the sample, wherein an increased level of PDCD2 protein or RNA in the sample as compared to the control indicates that the subject has cancer, or is at an elevated risk for developing cancer.

2. The method of claim 1, comprising quantifying the level of PDCD2 RNA.

3. The method of claim 2, wherein the level of PDCD2 RNA is quantified by reverse transcription polymerase chain reaction (RT-PCR), quantitative Real Time RT-PCR, Northern blot analysis, expression microarray analysis or next generation sequencing (NGS).

4. The method of claim 1, comprising quantifying the level of PDCD2 protein.

5. The method of claim 4, wherein the level of PDCD2 protein is quantified by Western analysis or immunohistochemistry of tissue microarrays.

6. The method of claim 1, wherein the cancer is acute leukemia.

7. The method of claim 1, wherein the cancer is a hematologic malignancy.

8. The method of claim 7, wherein the hematologic malignancy is selected from acute lymphoblastic leukemia, acute myelogenous leukemia, chronic lymphocytic leukemia, chronic myelogenous leukemia, advanced myelodysplasia, multiple myeloma and lymphoma.

9. The method of claim 1, wherein the cancer is carcinoma.

10. The method of claim 9, wherein the carcinoma is breast, ovarian, lung, gastrointestinal, or genitourinary carcinoma.

11. The method of claim 1, wherein the sample is peripheral blood, bone marrow or tissue.

12. The method of claim 1, wherein the subject has normal cytogenetics.

13. A method for determining the effectiveness of a cancer treatment in a subject, comprising:
a) quantifying the level of Programmed Cell Death 2 (PDCD2) protein or RNA in a first biological sample from the subject before the cancer treatment;
b) quantifying the level of Programmed Cell Death 2 (PDCD2) protein or RNA in a second biological sample from the subject after the cancer treatment;
c) comparing the level in the first sample to the level in the second sample; and
d) determining that the cancer treatment is effective based on the level of PDCD2 protein or RNA in the second sample compared to the first sample, wherein a decrease in the level of PDCD2 protein or RNA in the second sample as compared to the first sample indicates that the cancer treatment is effective to reduce the cancer.

14. The method of claim 13, wherein the level of PDCD2 RNA is quantified by reverse transcription polymerase chain reaction (RT-PCR), quantitative Real Time RT-PCR, Northern blot analysis, expression microarray analysis or next generation sequencing (NGS).

15. The method of claim 13, wherein the level of PDCD2 protein is quantified by Western analysis or immunohistochemistry of tissue microarrays.

16. A method for identifying a subject that has a relapse of cancer or refractory disease, or is at an elevated risk for a relapse of cancer or refractory disease, comprising:
a) quantifying the level of Programmed Cell Death 2 (PDCD2) protein or RNA in a first biological sample from the subject before a cancer treatment;
b) quantifying the level of Programmed Cell Death 2 (PDCD2) protein or RNA in a second biological sample from the subject after a cancer treatment; and
c) calculating the ratio of the level in the first sample to the level in the second sample, wherein a ratio less than about 2 indicates the subject has relapsed, or is at an elevated risk for a relapse of cancer or refractory disease.

17. The method of claim 16, wherein a ratio less than about 1.7 indicates the subject has relapsed or has refractory disease, or is at an elevated risk for a relapse of cancer or refractory disease.

18. The method of claim 16, wherein a ratio of about 1 indicates the subject has relapsed or has refractory disease, or is at an elevated risk for a relapse of cancer or refractory disease.

19. The method of claim 16, wherein the level of PDCD2 RNA is quantified by reverse transcription polymerase chain reaction (RT-PCR), quantitative Real Time RT-PCR, Northern blot analysis, expression microarray analysis or next generation sequencing (NGS).

20. The method of claim 16, wherein the level of PDCD2 protein is quantified by Western analysis or immunohistochemistry of tissue microarrays.
